(19) United States
(12) Patent Application Publication
(10) Pub. No.: US 2026/0355167 A1
(43) Pub. Date: Nov. 12, 2026

(54) Erodible Intraocular PKal Inhibitor Implants and Uses Thereof

(71) Applicant: Alcon Inc., Fribourg (CH)

(72) Inventors: David Archer Ellis, Durham, NC (US); Robert Vincent Brown, Durham, NC (US); Tyler James Pegoraro, Raleigh, NC (US); Asmerom O. Weldeab, Fuquay-Varina, NC (US)

(21) Appl. No.: 19/657,750

(22) Filed: Apr. 24, 2026

Related U.S. Application Data
(60) Provisional application No. 63/794,857, filed on Apr. 25, 2025.

(51) Int. Cl. A61K 31/4418; A61K 9/00; A61K 31/4439; A61K 38/17; A61K 39/395; A61K 47/34

(52) U.S. Cl. CPC A61K 31/4418; A61K 9/0024; A61K 9/0051; A61K 31/4439; A61K 38/179; A61K 39/3955; A61K 47/34

(57) ABSTRACT

Provided herein are compositions and methods for the treatment of retinal diseases, including diabetic macular edema, in a subject in need thereof. Compositions comprising a bioerodible intravitreal implant and PKal inhibitors are disclosed herein.

### RELATED APPLICATIONS

This application claims priority of U.S. Provisional Patent Application No. 63/794,857, filed Apr. 25, 2025, the entire content of which is incorporated herein by reference.

### BACKGROUND

Diabetic macular edema (DME) is a multifaceted disease that involves progressive vision impairment leading to legal or total blindness. Secondary to diabetes, the pathogenesis of DME is largely driven by vascular leakage, inflammation, and angiogenesis. The current standard of care is anti-VEGF (vascular endothelial growth factor) monotherapy. This one-dimensional approach primarily addresses angiogenesis. However, anti-VEGF monotherapy is a suboptimal treatment in about 50% of patients suffering from DME.

Plasma kallikrein (PKal) is a trypsin-like serine protease that is thought to play a role in several pathways relating to ocular inflammation and retinal vascular dysfunctions. Synthetic small molecule PKal inhibitors berotralstat and sebetralstat are orally bioavailable medicines clinically validated to reduce vascular leakage in patients suffering from hereditary angioedema (HAE).

Current retinal disease therapies may be delivered by intravitreal (IVT) injection (e.g., aflibercept (EYLEA®), bevacizumab (AVASTIN®), faricimab-svoa (VABYSMO®), or ranibizumab (LUCENTIS®)) or by IVT implant (e.g., dexamethasone (OZURDEX®) or fluocinolone acetonide (ILUVIEN®)). Due to a relatively short duration of action, however, IVT injection requires the patient to endure multiple ocular IVT injections over the course of therapy. This administration regimen is a stressful, uncomfortable, and an inconvenient burden on the patient. Additionally, IVT implant steroid therapy, such as OZURDEX® (CHAMPLAIN study) or ILUVIEN® (FAME study), has been associated with increased intraocular pressure (IOP) and cataract formation in a significant percentage of patients (Ehab N. El Rayes, “Into the Suprachoroidal Space,” Retina Today, January/February 2018). Other DME treatments include laser therapy (photocoagulation) or vitrectomy. Additionally, suprachoroidal space (SCS) injection has been proposed as an alternative tissue site for administration of steroids (Retina Today, 2018) or non-surgical administration of PKal inhibitors (US20220160694A1).

By contrast to these DME treatment options, provided herein are durable drug formulations, e.g., ocular implants, and delivery methods to improve outcomes including patient satisfaction, compliance, and therapeutic efficacy. According to the present disclosure, IVT implants are inserted by injection (e.g., surgical injection) for the treatment of retinal diseases.

Thus, some embodiments provided herein include polymer-based drug products in the form of bioerodible IVT implants, capable of tuned drug delivery to provide sustained release (e.g., maintenance of therapeutically effective levels of API, e.g., greater than in vitro IC₉₉, for about 6 months or more immediately subsequent to administration) of a plasma kallikrein (PKal) inhibitor or a PKal inhibitor/anti-VEGF combination product to the tissues of the back of the eye. This drug delivery approach provides improved efficacy, robustness, and durability for PKal or PKal/anti-VEGF drug delivery to DME patients, greatly reducing the need for multiple suprachoroidal space (SCS) or IVT injections.

### SUMMARY

It has been found that administering a pKal inhibitor into the vitreous humor is useful in treating retinal diseases, such as diabetic macular edema. The PKal inhibitor (e.g., avoralstat or a pharmaceutically acceptable salt thereof) may be paired with administration of a VEGF inhibitor (e.g., axitinib) into the vitreous humor.

Thus, in some embodiments the present disclosure describes implantable biodegradable ocular implants comprising a polymer matrix and PKal inhibitor or a PKal inhibitor/anti-VEGF combination product for administration in the vitreous to treat diabetic macular edema (DME). Some embodiments include a composition, comprising a polymeric matrix and a PKal inhibitor, wherein the PKal inhibitor is dispersed within the polymeric matrix, and the polymeric matrix is a bioerodible polymeric ocular implant. In some examples, the PKal inhibitor is berotralstat, sebetralstat, avoralstat, or a pharmaceutically acceptable salt thereof. In some embodiments, an implant comprising avoralstat is injected into the vitreous. Some examples include at least one compound selected, independently, from a vascular endothelial growth factor (VEGF) inhibitor or a vascular endothelial growth factor receptor (VEGFR) inhibitor. In some embodiments, the anti-VEGF compound may be dispersed within the polymeric matrix with the PKal inhibitor. In other embodiments, anti-VEGF compound may be administered alone or in its own delivery device at the same time and in conjunction with the PKal inhibitor composition described herein. In some embodiments, the anti-VEGF compound is axitinib, cabozantinib, fruquintinib, lenvatinib, pazopanib, ponatinib, regorafenib, sorafenib, sunitinib, or tivozanib, or a pharmaceutically acceptable salt thereof. In other embodiments, the anti-VEGF compound is aflibercept, bevacizumab, brolucizumab, faricimab, or ranibizumab, or a biosimilar thereof. Some embodiments include a polymeric matrix comprising a polyesteramide polymer and/or a poly(lactic-co-glycolic) acid (PLGA) polymer. In some examples, the polyesteramide (PEA) polymer is PEA III. In other examples, the PLGA polymer is a commercially available PLGA polymer. In some embodiments, a method of treating diabetic macular edema in a subject in need thereof is described. The method comprises administration of the implants described herein to a subject in need thereof via injection in the vitreous. In some examples, the subject experiences at least 10% improvement in measurement of a sign or symptom of diabetic macular edema as compared to IVT implant administration of dexamethasone (e.g., OZURDEX®) or fluocinolone acetonide (e.g., ILUVIEN®) to the subject or IVT injection of an anti-VEGF monotherapy (e.g., aflibercept (EYLEA®), bevacizumab (AVASTIN®), faricimab-svoa (VABYSMO®), ranibizumab (LUCENTIS®)) to the subject. In other examples, the implant releases the PKal inhibitor at an effective concentration over at least three months, or over at least six months.

These and other embodiments of the present disclosure are described in more detail below.

### BRIEF DESCRIPTION OF THE DRAWINGS

FIG. 1A depicts a polymer matrix and avoralstat composition described in the present disclosure.

FIG. 1B depicts implants prepared from a polymer matrix and avoralstat composition described in the present disclosure.

FIG. 1C depicts a polymer matrix and berotralstat composition described in the present disclosure.

FIG. 1D depicts implants prepared from a polymer matrix and berotralstat composition described in the present disclosure.

FIG. 1E depicts a polymer matrix and sebetralstat composition described in the present disclosure.

FIG. 1F depicts implants prepared from a polymer matrix and sebetralstat composition described in the present disclosure.

FIG. 2A depicts an in vivo rabbit ocular tissue pharmacokinetic (PK) profile following bolus intravitreal injection of sebetralstat.

FIG. 2B depicts an in vivo rabbit ocular tissue PK profile following bolus intravitreal injection of berotralstat.

FIG. 2C depicts an in vivo rabbit ocular tissue PK profile following bolus intravitreal injection of avoralstat.

FIG. 3A depicts an in vitro dissolution, in daily release amounts, of a PKal inhibitor from various polymer matrix formulations of the present disclosure.

FIG. 3B depicts an in vitro dissolution, in cumulative release amounts, of a PKal inhibitor from various polymer matrix formulations of the present disclosure.

FIG. 4 depicts an in vivo tissue concentration amount, for rabbit retinal tissue, of a PKal inhibitor from various polymer matrix formulations of the present disclosure.

FIG. 5A depicts an in vitro dissolution, in daily release amounts, of a PKal inhibitor from various polymer matrix formulations of the present disclosure.

FIG. 5B depicts an in vitro dissolution, in cumulative release amounts, of a PKal inhibitor from various polymer matrix formulations of the present disclosure.

FIG. 6 depicts an in vivo rabbit DL-AAA retinal vascular leakage model, using a PKal inhibitor and/or VEGF inhibitor in various polymer matrix formulations of the present disclosure.

FIG. 7 depicts an in vivo rabbit ocular tissue PK profile following intravitreal injection of an avoralstat implant.

FIG. 8 depicts an in vivo rabbit ocular tissue PK profile following intravitreal injection of an avoralstat implant.

FIG. 9 depicts an in vivo rabbit ocular tissue PK profile following intravitreal injection of an avoralstat implant.

FIG. 10 depicts an in vivo tissue concentration amount, in rabbit retinal tissue, of avoralstat from various implant formulations of the present disclosure.

FIG. 11 depicts an in vivo tissue concentration amount, in rabbit retinal pigmented epithelium (RPE) tissue, of avoralstat from various implant formulations of the present disclosure.

### DETAILED DESCRIPTION

The present disclosure describes intravitreal implant compositions comprising a polymer matrix and a PKal inhibitor (e.g., avoralstat or a pharmaceutically acceptable salt thereof). The present disclosure also includes intravitreal implant compositions comprising a polymer matrix, a PKal inhibitor (e.g., avoralstat or a pharmaceutically acceptable salt thereof), and an anti-VEGF compound (e.g., axitinib). The PKal inhibitor and/or the anti-VEGF compound are the active pharmaceutical ingredient (API) of the intravitreal implant compositions described herein. The compositions of the present disclosure are designed to be administered intravitreally (e.g., surgically) for the treatment of eye diseases, including diabetic retinopathy (DR), proliferative diabetic retinopathy (PDR), non-proliferative diabetic retinopathy (NPDR), macular edema (e.g., diabetic macular edema (DME)), wet age-related macular degeneration (wet AMD), or retinal vein occlusion (RVO).

### DEFINITIONS

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, the term “about” may explicitly or implicitly modify all numerical values provided herein, unless otherwise indicated.

The term “about” generally indicates a possible variation of a numerical value. In some embodiments, the possible variation (±) may be no more than 10%, 5%, or 1% of the numerical value. In some embodiments, the last decimal place of a numerical value provided herein indicates its degree of accuracy. In some embodiments, where no other error margins are given, the maximum margin is ascertained by applying the rounding-off convention to the last decimal place or last significant digit when a decimal is not present in the given numerical value.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “biodegradable” or “bioerodable” or “erodible” mean that at least the implants here are capable of being broken down into innocuous and bioactive products in the normal functioning of the body. The biodegradable polymers used herein have hydrolysable ester linkages, which provide the biodegradability and are typically chains bearing carboxyl groups.

The terms “composition” and “pharmaceutical composition” refer to a mixture of at least one compound described herein with another component, such as a carrier or a pharmaceutically acceptable carrier, respectively. The pharmaceutical composition facilitates administration of the compound to a patient or subject.

The terms “effective amount” and “therapeutically effective amount” refer to an amount of therapeutic compound, such as a compound described herein, administered to a subject, either as a single dose or as part of a series of doses, which is effective to produce a desired therapeutic effect.

The term “pharmaceutically acceptable carrier” means a pharmaceutically acceptable material, composition, or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions or dosage forms described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its ionized form and paring the ionized parent compound with a counterion to form a salt form. Lists of salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The term “solid form” includes, but is not limited to, polymorphs, crystalline forms, amorphous forms, solvates, hydrates, or co-crystals of a compound or a salt thereof.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Ratios of polymers in the figures are depicted as weight percentages. By way of example, 7505E/X25 (90/10) refers to a polymer matrix having about 90% by weight polymer 7505E and about 10% by weight polymer X25, and Avoralstat/7505E/5005A (40%/42%/18%) refers to a polymer matrix having about 70% by weight polymer 7505E and about 30% by weight polymer 5005A where the polymer matrix is about 60% by weight of the implant composition. Other polymer ratio depictions herein are similarly understood.

Recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order, and may include a combination of one or more embodiments herein, unless otherwise indicated herein or otherwise clearly contradicted by context. The use of examples, or exemplary language (“for example,” “such as,” etc.) provided herein is intended merely to better illuminate the described subject matter and does not pose a limitation on the scope of the subject matter otherwise claimed. No language in the specification should be construed as indicating any non-claimed element essential to practicing the described subject matter.

Each group member of a grouping of alternative elements or embodiments of this disclosure may be referred to and claimed individually or in any combination with other members of the group or other elements found herein. Furthermore, a recited member of a group may be included in, or excluded from, another recited group for reasons of convenience or patentability.

The contents of all references (including literature references, issued patents, published patent applications, and co-pending patent applications) cited throughout this application are hereby expressly incorporated herein by reference in their entireties.

Unless otherwise defined, all technical and scientific terms used herein are accorded the meaning commonly known to one with ordinary skill in the art.

Embodiments of this disclosure are illustrative. Accordingly, the present disclosure is not limited to that precisely as shown and described.

### Retinal Vascular Diseases Pathogenic Mechanisms

Wet AMD, DR, NPDR, PDR, DME and RVO are distinct retinal vascular diseases that share a common pathogenic framework, microvascular injury, ischemia, vascular permeability, inflammation, and neurovascular unit dysfunction. At the foundational level vascular leakage and aberrant angiogenesis drive vision threatening pathologies in these diseases. Microvascular exudation and leakage follow retinal capillary dysfunction leading to ischemia and hypoxia. Hypoxic tissues produce potent pro-angiogenic signaling factors including the soluble extracellular signaling protein vascular endothelial growth factor (VEGF-A, -B, -C, -D, -E, or -F). VEGF promotes endothelial cell proliferation by binding to its cognate receptor, VEGF-R (−1, −2, or −3), serving as a master regulator of angiogenesis in retinal vascular disease. Accordingly, the VEGF signaling axis is an effective target for treating retinal vascular disease by either targeted neutralization of the VEGF ligand (e.g., aflibercept) or by inhibition of VEGF-R kinase activity (e.g., axitinib).

Though effective in treating retinal vascular disorders in many patients, some 30-50% of patients treated with anti-VEGF therapies are sub-optimally treated and maintain vision threatening disease progression. Targeted inhibition of the PKal pathway represents an effective way to manage VEGF-independent contributions to retinal vascular disease and has potential for additive or synergistic therapeutic outcomes.

### DME

VEGF is known to be a key mediator in DME pathogenesis, and VEGF inhibitors are the standard of care for DME. Anti-VEGF intravitreal therapy is the first-line treatment for DME. PKal is known to play a role in DME pathogenesis. DME may have a serious impact on a subject's quality of life. Signs and symptoms of DME include blurry vision, double vision, floaters, difficulty seeing colors, dark spots, distorted central vision, difficulty reading, loss of contrast, or blindness. In some embodiments of the present disclosure, the implants herein injected in the vitreous may result in an improvement in one or more of the signs and symptoms of DME. In some embodiments, a 10% improvement in one or more of the signs or symptoms of DME may be achieved. In other embodiments, an improvement of greater than 10% in one or more of the signs or symptoms of DME may be achieved. Some examples may provide up to a 50% improvement in one or more of the signs or symptoms of DME.

### Plasma Kallikrein Inhibitors and Retinal Vascular Diseases

The implant compositions of the present disclosure comprise an active pharmaceutical ingredient (API) dispersed (e.g., homogeneously) within a polymeric matrix. In some embodiments, the API is a plasma kallikrein (referred to hereinafter as “PKal”) inhibitor. PKal is a trypsin-like serine protease that is the sole enzyme responsible for enzymatic cleavage of kininogen to high molecular weight kininogen (HMWK), which liberates bradykinin leading to increased vascular permeability.

The plasma kallikrein (PKal) pathway is a VEGF-independent contributor to retinal vascular pathology responsible for neurovascular dysfunction, leakage, and inflammation. Retinal capillary damage and aberrant exudation/leakage of blood-plasma proteins activate PKal leading to the aberrant production of bradykinin. Unregulated production of bradykinin in the retina causes inflammation, sustained vascular leakage, and edema. This mechanism is a driving cause of the genetic disease known as hereditary angioedema (HAE) which is effectively treated by PKal inhibitors. The plasma kallikrein-kinin system is abnormally abundant in patients with advanced DME. PKal contributes to retinal vascular dysfunctions in diabetic rats (A. Clermont et al., “Plasma kallikrein mediates retinal vascular dysfunction and induces retinal thickening in diabetic rats” Diabetes. 2011, 60, 1590-1598). Furthermore, administration of the PKal inhibitor ASP-440 ameliorated both retinal vascular permeability and retinal blood flow abnormalities in diabetic rats.

Provided herein are PKal inhibitors (e.g., avoralstat or a pharmaceutically acceptable salt thereof) useful in treatments to reduce retinal vascular permeability associated with retinal vascular diseases and DME in particular. The treatment, inhibition, or amelioration of one or more ophthalmic diseases associated with PKal, including HAE, uveitis, posterior uveitis, wet AMD, DR, NPDR, PDR, and RVO may also be considered as targets for a PKal inhibitor.

Synthetic and small molecule PKal inhibitors have been described, and two of these have been approved as oral products for medical use. These are berotralstat (“Berotralstat (BCX7353): Structure-Guided Design of Potent, Selective, and Oral Plasma Kallikrein Inhibitor to Prevent Attacks of Hereditary Angioedema (HAE)”, J. Med. Chem. 2021, 64, 12453-12468) and sebetralstat (“Sebetralstat (KVD900): A Potent and Selective Small Molecule Plasma Kallikrein Inhibitor Featuring a Novel P1 Group as a Potential Oral On-Demand Treatment for Hereditary Angioedema”, J. Med. Chem. 2022, 65, 13629-13644).

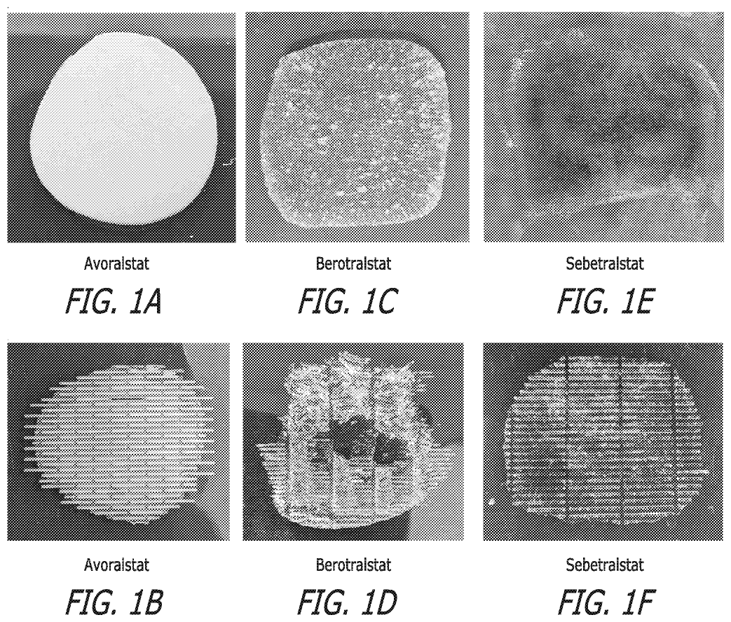

The implants of the present disclosure may comprise any suitable PKal inhibitor compound, though it has been found that certain polymer matrices combined with specific PKal inhibitors may, under some conditions, not provide a useable intravitreal implant (see FIG. 1C, FIG. 1D, FIG. 1E, and FIG. 1F). In some embodiments, the PKal inhibitor is in a solid form. PKal inhibitor compounds utilized herein can include avoralstat, or a pharmaceutical salt/co-crystal thereof. Some embodiments include avoralstat as the PKal inhibitor. The structures of avoralstat, berotralstat, and sebetralstat are shown below.

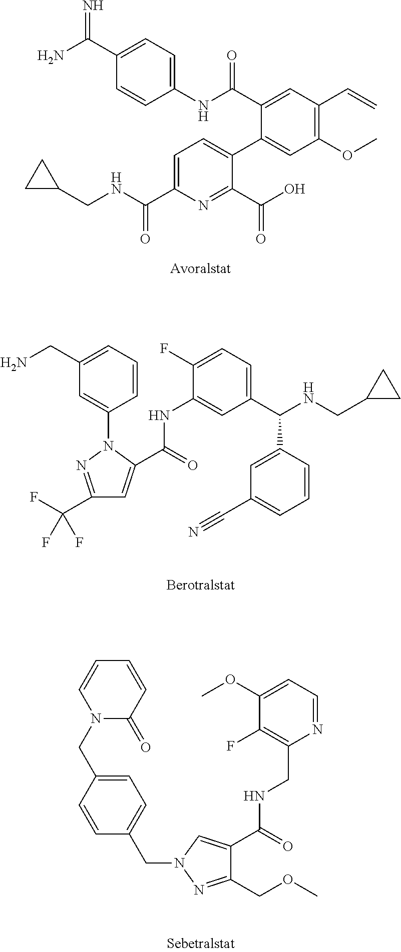 [SMILES: C=Cc1cc(C(=O)Nc2ccc(C(=N)N)cc2)c(-c2ccc(C(=O)NCC3CC3)nc2C(=O)O)cc1OC ; N#Cc1cccc([C@@H](NCC2CC2)c2ccc(F)c(NC(=O)c3cc(C(F)(F)F)nn3-c3cccc(CN)c3)c2)c1 ; COCc1nn(Cc2ccc(Cn3ccccc3=O)cc2)cc1C(=O)NCc1nccc(OC)c1F]

Any appropriate weight amount of the PKal inhibitor may be used in the compositions of the present invention. In some examples, the relative weight percentage of the PKal inhibitor in the polymer matrix may be guided by in vitro inhibition assays. In some embodiments, the IC₉₉ of the PKal inhibitor may be used to select an appropriate relative weight of the PKal inhibitor in the embodiments. In other examples, the amount (and nature of) the PKal inhibitor used is guided by the in vitro dissolution of the PKal inhibitor. In some embodiments, the weight of the PKal inhibitor (e.g., avoralstat or a pharmaceutically acceptable salt thereof) in the implant may be about 16 mg to about 660 mg, about 34-90 mg, about 60-160 mg, about 82-328 mg, about 250-500 mg, about 300-450 mg, or about 45 mg, about 56 mg, about 60 mg, about 68 mg, about 80 mg, about 90 mg, about 100 mg, about 120 mg, about 140 mg, about 160 mg, about 170 mg, about 180 mg, about 190 mg, about 200 mg, about 250 mg, about 330 mg, about 340 mg, about 350 mg, about 360 mg, about 370 mg, about 380 mg, about 390 mg, about 400 mg, about 410 mg, about 420 mg, about 430 mg, or any weight in a range bounded by any of these values.

Thus, some embodiments provided herein include polymer-based drug products in the form of bioerodible IVT implants, capable of tuned drug delivery to provide sustained release of a plasma kallikrein (PKal) inhibitor or a PKal inhibitor/anti-VEGF combination product to the tissues of the back of the eye. In some embodiments, sustained release refers to maintenance of therapeutically effective levels of or sustained levels of API, e.g., greater than in vitro IC₉₉ of the API, for about 6 months or more, immediately subsequent to administration. In some embodiments, the implants herein, such as those including avoralstat or a pharmaceutically acceptable salt thereof, are configured to provide a sustained release of avoralstat or a pharmaceutically acceptable salt thereof for about 6 months or more. In some embodiments, the implants herein, such as those including avoralstat or a pharmaceutically acceptable salt thereof, are configured to provide a sustained release of avoralstat or a pharmaceutically acceptable salt thereof for about 6 months or more such that the retinal tissue concentration of avoralstat or a pharmaceutically acceptable salt thereof is about 80 nM or more for about 6 months or more. Thus, in some embodiments, the implants herein are configured to include about 20-50% w/w (e.g., about 20% w/w, about 30% w/w, about 40% w/w, or about 50% w/w) avoralstat or a pharmaceutically acceptable salt thereof and about 50-80% w/w (e.g., about 50% w/w, about 60% w/w, about 70% w/w, or about 80% w/w) polymer matrix.

Thus, in some embodiments, provided herein is an intravitreal implant composition (e.g., a single implant) comprising: about 20-50% w/w of a PKal inhibitor (e.g., avoralstat or a pharmaceutically acceptable salt thereof); and about 50-80% w/w polymer matrix; wherein the composition comprises about 16 mg to about 660 mg of the PKal inhibitor, and optionally the implant is configured for administration by injection through a needle (e.g., a 23 or 25 gauge needle).

In some embodiments, the intravitreal implant composition comprises (e.g., is a single implant comprising) about 35-55% (e.g., about 40%) w/w of a PKal inhibitor (e.g., avoralstat or a pharmaceutically acceptable salt thereof) and about 55-65% (e.g., about 60%) w/w polymer matrix; wherein the composition comprises about 300 mg to about 500 mg (e.g., about 350-450 mg) of the PKal inhibitor, and optionally the implant is configured for administration by injection through a needle (e.g., a 23 gauge needle).

In some embodiments, the intravitreal implant composition comprises (e.g., is a single implant comprising) about 35-55% (e.g., about 40%) w/w of a PKal inhibitor (e.g., avoralstat or a pharmaceutically acceptable salt thereof) and about 55-65% (e.g., about 60%) w/w polymer matrix; wherein the composition comprises about 100 mg to about 300 mg (e.g., about 150-250 mg) of the PKal inhibitor, and optionally the implant is configured for administration by injection through a needle (e.g., a 25 gauge needle).

### VEGF and VEGFR Inhibitors

In addition to a PKal inhibitor, the implant compositions of the present disclosure optionally further comprise an anti-VEGF compound. For the purposes of the present disclosure, the VEGF inhibitors and VEGFR inhibitors described herein are also termed “anti-VEGF” compounds. Vascular endothelial growth factor (VEGF) is an important signaling protein involved in both vasculogenesis (the formation of the circulatory system) and angiogenesis (the growth of blood vessels from pre-existing vasculature). The normal function of VEGF is to create new blood vessels during embryonic development, new blood vessels after injury, muscle following exercise, and new vessels (collateral circulation) to bypass blocked vessels. VEGF can contribute to disease. Solid cancers cannot grow beyond a limited size without an adequate blood supply; cancers that can express VEGF are able to grow and metastasize. Overexpression of VEGF can cause vascular disease, including DME, in the retina of the eye and other parts of the body. Drugs that inhibit VEGF can control or slow those diseases. Many VEGF inhibitory compounds are known, and any suitable anti-VEGF compound may be selected for use in the present disclosure. In some embodiments, the anti-VEGF compound may be a small molecule kinase inhibitor selected from axitinib, cabozantinib, fruquintinib, lenvatinib, pazopanib, ponatinib, pegaptanib, regorafenib, sorafenib, sunitinib, or tivozanib, or a pharmaceutically acceptable salt thereof. In other embodiments, the anti-VEGF compound may be aflibercept, bevacizumab, brolucizumab, faricimab, or ranibizumab, or a biosimilar thereof. Any appropriate weight amount of the anti-VEGF compound may be used in the compositions of the present invention. Some embodiments include an implant comprising a polymer matrix and a PKal inhibitor to be administered to a subject in need thereof. In some embodiments, both the PKal inhibitor and the anti-VEGF compound may be incorporated into the polymer matrix of the implant to be administered to a subject in need thereof. In other embodiments, the anti-VEGF compound (alone, or incorporated into its own implant) may be administered separately from, but in conjunction with, an implant containing the polymer matrix and the PKal inhibitor.

### Intravitreal Implant Compositions

The intravitreal implant compositions provided herein include a polymer matrix and one or more API (e.g., PKal inhibitor, e.g., avoralstat or a pharmaceutically acceptable salt thereof). In some embodiments of the intravitreal implant compositions provided herein, the intravitreal implant compositions comprise about 20-50% w/w one or more API and about 50-80% w/w polymer matrix. In some embodiments of the intravitreal implant compositions provided herein, the intravitreal implant compositions comprise about 20-40% w/w one or more API and about 60-80% w/w polymer matrix. In some embodiments of the intravitreal implant compositions provided herein, the intravitreal implant compositions comprise about 50% w/w one or more API and about 50% w/w polymer matrix. In some embodiments of the intravitreal implant compositions provided herein, the intravitreal implant compositions comprise about 45% w/w one or more API and about 55% w/w polymer matrix. In some embodiments of the intravitreal implant compositions provided herein, the intravitreal implant compositions comprise about 40% w/w one or more API and about 60% w/w polymer matrix. In some embodiments of the intravitreal implant compositions provided herein, the intravitreal implant compositions comprise about 35% w/w one or more API and about 65% w/w polymer matrix. In some embodiments of the intravitreal implant compositions provided herein, the intravitreal implant compositions comprise about 30% w/w one or more API and about 70% w/w polymer matrix. In some embodiments of the intravitreal implant compositions provided herein, the intravitreal implant compositions comprise about 25% w/w one or more API and about 75% w/w polymer matrix. In some embodiments of the intravitreal implant compositions provided herein, the intravitreal implant compositions comprise about 20% w/w one or more API and about 80% w/w polymer matrix.

In some embodiments, the polymer matrix comprises one or more than one, e.g., a first and a second, polymer. Suitable polymers include, but are not limited to, polyethylenes, polyacrylates, polymethacrylates, polyesters, polyamides, polyesteramides (PEA), poly(lactic-co-glycolic) acids (PLGA), poly(lactic) acids (PLA), polycarbonates, polyurethanes, polyanhydrides, polyketones, polyphenols, and the like. In some embodiments, the one (i.e., single polymer in the polymer matrix) or first polymer is a PEA, PLGA, or PLA polymer and the second polymer, different from the first polymer, is a PLGA or a PLA polymer. In some embodiments, the polymer matrix comprises a third polymer, different from the first and second polymers, selected from a PLGA or a PLA polymer. In some embodiments, the intravitreal implant compositions comprise the first and second polymers in about a 2:3 to 4:1 ratio (e.g., about 2:3, 1:1, 3:2, 7:3, or 4:1).

In some embodiments, polyesteramide (PEA) copolymers may be included in the polymer matrices. Some examples include α-amino acid-diol-diester containing PEA (e.g., G. Tsitlanadze, et al. J. Biomater. Sci. Polym. Edn. (2004) 15:1-24). These PEA polymers show enzyme-mediated surface degradation and a low inflammation profile (K. DeFife et al., Transcatheter Cardiovascular Therapeutics-TCT 10 2004 Conference). Any suitable α-amino acid may be chosen. In some embodiments, the naturally occurring α-amino acid comprises alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or a combination thereof. In some embodiments, the α-amino acid comprises leucine and lysine. In some embodiments, a functional group (e.g., a hydroxyl group, a thiol group, an amino group, or a carboxylic acid group) of an α-amino acid used herein may be further functionalized. Some embodiments include a lysine moiety with its carboxylic acid functionalized as a benzyl ester to modulate the physical properties of polymer, implant matrix, and ultimately drug release profile. In some embodiments, the PEA grades include ratios for pendant lysine carboxyl groups in the form of free carboxyl groups versus benzyl ester carboxyl groups ranging from 1:4 to 3:4. In some embodiments, the molecular weight for PEA includes ranges from 20-75 kDa.

In other embodiments, poly(lactic-co-glycolic) acid (PLGA) may be included in the polymer matrices. PLGA is a copolymer which is used in a host of Food and Drug Administration (FDA) approved therapeutic devices. PLGA is synthesized by means of ring-opening co-polymerization of two different monomers, the cyclic dimers (1,4-dioxane-2,5-diones) of glycolic acid and lactic acid. Thus, PLGA polymers comprise a mole fraction of lactic acid and a mole fraction of glycolic acid. Polymers can be synthesized as either random or block copolymers thereby imparting additional polymer properties. In addition, the relative amounts of glycolic acid and lactic acid monomer can be adjusted to afford PLGA's with certain desired physical properties (including degradation properties). Further, PLGAs that are end-capped with esters (e.g., methyl esters, as opposed to the free carboxylic acid) demonstrate longer degradation half-lives. PLGA with different molecular weight can also be used to modulate physical properties of the implants referred to herein. Each of these facets may be used to generate a PLGA with properties tailored to a given application or tailored to a particular API. Many PLGAs are commercially available. Examples include PLGA Resomer® 502, 503, 653, 752, etc., which the first two numbers relate to the percentage of lactic acid in the polymer and the last number relates to the molecular weight. Other examples include PLGA Viatel™ 5003, 5005, 6503, 7503, etc., which the first two numbers relate to the percentage of lactic acid in the polymer and the last two numbers relates to the molecular weight. Common designations include “H” or “A” for acid terminated (e.g., 502H or 5003A), and “S” or “E” for ester terminated PLGAs (e.g., 502S or 5003E).

It has been found that the release rate of a PKal inhibitor (e.g., avoralstat or a pharmaceutically acceptable salt thereof) is too rapid, for purposes of therapeutic efficacy lasting at least 6 months using the intravitreal implant compositions herein, when the mole fraction of lactic acid in the PLGA is less than the mole fraction of glycolic acid in the PLGA. It has been found that the release rate of a VEGF inhibitor (e.g., axitinib) is too rapid, for purposes of therapeutic efficacy lasting at least 6 months using the intravitreal implant compositions herein, when the mole fraction of lactic acid in the PLGA is less than the mole fraction of glycolic acid in the PLGA.

Thus, in some embodiments, the PLGA polymer, when included in a polymer matrix herein, has a mole fraction of lactic acid of not less than the mole fraction of glycolic acid. In other embodiments, the PLGA polymer has a mole fraction of lactic acid of greater than 50% and a mole fraction of glycolic acid of less than 50%. In some embodiments, the PLGA polymer has a mole fraction of lactic acid of about 65% and a mole fraction of glycolic acid of about 35%. Some PLGA embodiments include a mole fraction of lactic acid of about 75% and a mole fraction of glycolic acid of about 25%. Other PLGA embodiments include a mole fraction of lactic acid of about 85% and a mole fraction of glycolic acid of about 15%. In some embodiments, the PLGA polymer has a mole ratio of lactic acid to glycolic acid ranging from 1:1 to about 17:3.

In other embodiments, poly(lactic) acid (PLA) may be included in the polymer matrices. Some embodiments of the polymer matrix include at least 10% of a PLA polymer. In some embodiments, the molecular weight for PLA includes ranges from 15-40 kDa.

Thus, in some embodiments, the polymer matrix comprises 0-30% w/w of a first polymer component and 70-100% w/w of a second polymer component. In other embodiments, the polymer matrix comprises 0-50% w/w of a first polymer component and 50-100% w/w of a second polymer component. In some embodiments, the polymer matrix comprises 0-10% w/w of a first polymer component and 90-100% w/w of a second polymer component. In other embodiments, the polymer matrix comprises 0-20% w/w of a first polymer component and 80-100% w/w of a second polymer component. In some embodiments, the polymer matrix comprises 0-40% w/w of a first polymer component and 60-100% w/w of a second polymer component.

In some embodiments, the intravitreal implant composition comprises avoralstat or a pharmaceutically acceptable salt thereof, and a polymer matrix comprising 0-30% w/w of a first polymer component and 70-100% w/w of a second polymer component. In other embodiments, the intravitreal implant composition comprises avoralstat or a pharmaceutically acceptable salt thereof, and a polymer matrix comprising 0-50% w/w of a first polymer component and 50-100% w/w of a second polymer component. In some embodiments, the intravitreal implant composition comprises avoralstat or a pharmaceutically acceptable salt thereof, and a polymer matrix comprising 0-10% w/w of a first polymer component and 90-100% w/w of a second polymer component. In other embodiments, the intravitreal implant composition comprises avoralstat or a pharmaceutically acceptable salt thereof, and a polymer matrix comprising 0-20% w/w of a first polymer component and 80-100% w/w of a second polymer component. In some embodiments, the intravitreal implant composition comprises avoralstat or a pharmaceutically acceptable salt thereof, and a polymer matrix comprising 0-40% w/w of a first polymer component and 60-100% w/w of a second polymer component.

In some embodiments, the intravitreal implant composition comprises avoralstat or a pharmaceutically acceptable salt thereof, and a polymer matrix comprising 100% w/w PEA III X50. In other embodiments, the intravitreal implant composition comprises avoralstat or a pharmaceutically acceptable salt thereof, and a polymer matrix comprising 100% w/w PEA III X75.

In some embodiments, the first polymer component comprises a PEA or a combination of PEAs, such as those of Table 1, e.g., PEA III X25, PEA III X35, PEA III X50, PEA III X65, or PEA III X75. In some embodiments, the first polymer component comprises or consists of PEA III X50. In some embodiments, the polymer matrix comprises or consists of PEA III X50. In some embodiments, the first polymer component comprises or consists of PEA III X25. In other embodiments, the first polymer component comprises or consists of PEA III X75.

In some embodiments, the second polymer component comprises a PLA or a combination of different PLAs, such as those of Table 3, e.g., DL 03 A or DL 03 E.

In some embodiments, the second polymer component comprises a PLGA or a combination of different PLGAs, such as those of Table 2, Table 3, or Table 4.

In some embodiments, the second polymer component comprises a combination of PLA and PLGA. In other embodiments, the second polymer component comprises a combination of PEA and PLGA.

Some embodiments of the present disclosure include a combination of PEA polymers and PLGA polymers to form a polymer matrix of PEA/PLGA. Like the individual components of the PEA and the PLGA as described above, the relative amounts of PEA and PLGA can also be adjusted to arrive at a desired rate of release of the API component of the implant. In some embodiments, the implants of the present invention are designed to release a PKal inhibitor (or a PKal inhibitor and an anti-VEGF compound) from the polymeric implant at a rate designed to achieve IC₉₉ levels in retinal target tissue for a specified period (e.g., at least half of a year, e.g., about 6 months, e.g., about half a year up to about one year, e.g., about 6 months to about 12 months). In some embodiments, this period may be at least 1 month, at least 2 months, at least 3 months, at least 4 months, at least 5 months, at least 6 months, or longer than 6 months. In some embodiments, an implant may release 10 times the IC₉₉ for 180 days. In other embodiments, the implant may release 10 times the IC₉₉ for 90 days. In some embodiments, a PEA/PLGA polymer matrix may be composed of varying amounts of each polymer. Some embodiments include a polymer matrix comprising 70% PEA III X50 and 30% PLGA RG 752S. In other embodiments, a polymer matrix of 90% PEA III X50 and 10% PLGA RG 502 is used as the polymer matrix. In still other embodiments, a polymer matrix of 70% PEA III X50, 15% PLGA RG 752S and 15% PLGA RG 653H is employed.

Thus, in some embodiments, the polymer matrix comprises a first API component and a polymer matrix, and optionally comprises a second API component different from the first API component. In some embodiments the first API component comprises a PKal inhibitor compound. In some embodiments, the first API component comprises an anti-VEGF compound. In some embodiments, the first API component comprises a PKal inhibitor compound and the second API component comprises an anti-VEGF compound. In some embodiments, the polymer matrix comprises a PEA, a PLGA, a blend of different PLGAs, or a blend of PEA and one or more PLGAs.

In some embodiments, the polymeric component of the polymer matrix comprises a blend (e.g., homogeneous blend) of a PEA, a first PLGA, and a second PLGA that is different from the first PLGA. In some embodiments, the PEA is selected from PEA, PEA III X25, PEA III X35, PEA III X50, PEA III X65, or PEA III X75, the first PLGA is selected from RG502, RG502H, RG503, RG503H, RG504, RG504H, RG505, RG505H, RG653, RG653H, RG752, RG752H, RG756, RG756H, RG858, or RG858H, and the second PLGA, which is different from the first PLGA, is selected from RG502, RG502H, RG503, RG503H, RG504, RG504H, RG505, RG505H, RG653, RG653H, RG752, RG752H, RG756, RG756H, RG858, or RG858H. The formula for PEAs or PLGAs referred to herein is briefly described in Table 1, Table 2, Table 3, and Table 4 below.

In some embodiments, the first API component of the polymer matrix comprises a PKal inhibitor, which is selected from avoralstat, or a pharmaceutically acceptable salt thereof. In some embodiments, the optional second API component of the polymer matrix comprises an anti-VEGF compound. In some embodiments, the second API component of the polymer matrix comprises a VEGF inhibitor. In some embodiments, the second API component of the polymer matrix comprises a VEGFR inhibitor. In some embodiments, the second API component of the polymer matrix is selected from axitinib, cabozantinib, fruquintinib, lenvatinib, pazopanib, ponatinib, regorafenib, sorafenib, sunitinib, or tivozanib, or a pharmaceutically acceptable salt thereof. In some embodiments, the second API component of the polymer matrix is selected from aflibercept, bevacizumab, brolucizumab, faricimab, or ranibizumab, or a biosimilar thereof.

In some embodiments, the intravitreal implant compositions herein are prepared by a process comprising a thermal melt step. The thermal melt step includes heating the polymer matrix and API to a temperature suitable to melt the polymer matrix (e.g., at about 150 °C to about 200 °C, e.g., about 150-180 °C, e.g., about 180 °C), then cooling the molten mixture in a desired shape and size such that the intravitreal implant is prepared. In some embodiments, the melting, cooling, and shaping of the implant is accomplished by thermal melt extrusion, e.g., extruding the molten mixture through an aperture and cooling and cutting the extruded composition to form a shaped IVT implant. In some embodiments, the melting, cooling, and shaping of the implant is accomplished by forming the molten mixture in a mold and cooling the composition to prepare the shaped IVT implant. It has been discovered that avoralstat, but not sebetralstat or berotralstat, keeps its physical form and remains chemically stable during manufacturing making it the only one amenable to these types of thermal melt implant manufacturing process. In some embodiments, the polymer(s) and the API(s) are combined and an intravitreal implant herein is prepared by a thermal melt manufacturing process that includes a thermal melt step. In some embodiments, the thermal melt step includes heating the combined polymer(s) and API(s) to a certain minimum temperature suitable to melt the polymer yet retain the chemical stability of the API(s) (e.g., the purity of the API(s)) or the physical form of the API(s), or both, after thermal melt manufacture formation of the intravitreal implant. In some embodiments, the purity of the API(s) in the formed intravitreal implant is not less than about 95% (e.g., not less than about 99%) of the input API(s) (i.e., the API(s) prior to blending and melting with the polymer(s)).

In some embodiments herein, the intravitreal implant composition comprises:
A) 60% w/w PEA III X50 and 40% w/w PKal inhibitor (e.g., avoralstat or a pharmaceutically acceptable salt thereof);
B) 42% w/w PEA III X50, 18% w/w PLGA 752S, and 40% w/w PKal inhibitor (e.g., avoralstat or a pharmaceutically acceptable salt thereof);
C) 30% w/w PLGA 503, 30% PLGA 503H, and 40% w/w PKal inhibitor (e.g., avoralstat or a pharmaceutically acceptable salt thereof);
D) 60% w/w PEA III X25 and 40% w/w PKal inhibitor (e.g., avoralstat or a pharmaceutically acceptable salt thereof); or
E) 42% w/w PEA III X25, 18% w/w PLGA 752S, and 40% w/w PKal inhibitor (e.g., avoralstat or a pharmaceutically acceptable salt thereof).

In some embodiments herein, the intravitreal implant composition comprises 40% w/w PKal inhibitor (e.g., avoralstat or a pharmaceutically acceptable salt thereof) and 60% w/w of the polymer(s) of the compositions described in Table 4.

TABLE 1
Structures of certain PEA polymers herein.

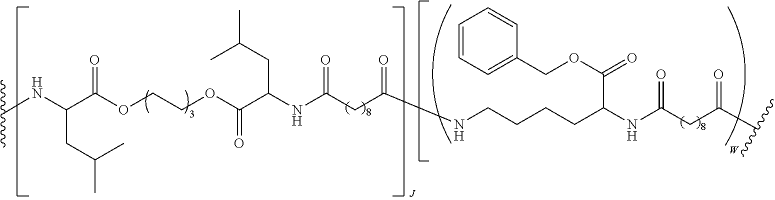

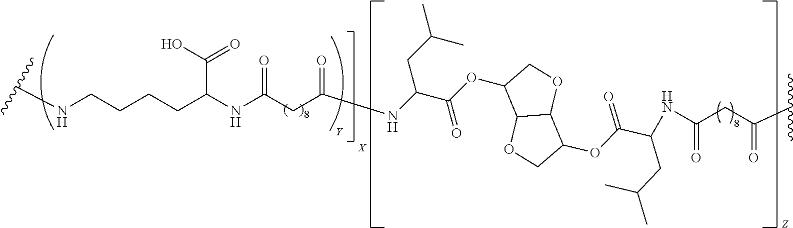

| | J | X | Z |
|---|---|---|---|
| PEA | 30% | 25% (W:Y is | 45% |
| | | 100:0) | |
| PEA III X25 | 30% | 25% (W:Y is | 45% |
| | | 75:25) | |
| PEA III X35 | 30% | 25% (W:Y is | 45% |
| | | 65:35) | |
| PEA III X50 | 30% | 25% (W:Y is | 45% |
| | | 50:50) | |
| PEA III X65 | 30% | 25% (W:Y is | 45% |
| | | 35:65) | |
| PEA III X75 | 30% | 25% (W:Y is | 45% |
| | | 25:75) | |

TABLE 2
Description of certain PLGA polymers herein.

| | |
|---|---|
| RG 502S, Poly(D,L-Lactide-co-Glycolide) | lactide:glycolide 50:50, ester |
| | terminated, M_{w} 7,000-17,000 |
| RG 502H, Poly(D,L-Lactide-co-Glycolide) | lactide:glycolide 50:50, acid terminated, |
| | M_{w} 7,000-17,000 |
| RG 503S, Poly(D,L-Lactide-co-Glycolide) | lactide:glycolide 50:50, ester |
| | terminated, M_{w} 24,000-38,000 |
| RG 503H, Poly(D,L-Lactide-co-Glycolide) | lactide:glycolide 50:50, acid terminated, |
| | M_{w} 24,000-38,000 |
| RG 504S, Poly(D,L-Lactide-co-Glycolide) | lactide:glycolide 50:50, ester |
| | terminated, M_{w} 38,000-54,000 |
| RG 504H, Poly(D,L-Lactide-co-Glycolide) | lactide:glycolide 50:50, acid terminated, |
| | M_{w} 38,000-54,000 |
| RG 505S, Poly(D,L-Lactide-co-Glycolide) | lactide:glycolide 50:50, ester |
| | terminated, M_{w} 54,000-69,000 |
| RG 505H, Poly(D,L-Lactide-co-Glycolide) | lactide:glycolide 50:50, acid terminated, |
| | M_{w} 54,000-69,000 |
| RG 653S, Poly(D,L-Lactide-co-Glycolide) | lactide:glycolide 65:35, ester |
| | terminated, M_{w} 24,000-38,000 |
| RG 653H, Poly(D,L-Lactide-co-Glycolide) | lactide:glycolide 65:35, acid terminated, |
| | M_{w} 24,000-38,000 |
| RG 752S, Poly(D,L-Lactide-co-Glycolide) | lactide:glycolide 75:25, ester |
| | terminated, M_{w} 4,000-15,000 |
| RG 752H, Poly(D,L-Lactide-co-Glycolide) | lactide:glycolide 75:25, acid terminated, |
| | M_{w} 4,000-15,000 |
| RG 756S, Poly(D,L-Lactide-co-Glycolide) | lactide:glycolide 75:25, ester |
| | terminated, M_{w} 76,000-115,000 |
| RG 756H, Poly(D,L-Lactide-co-Glycolide) | lactide:glycolide 75:25, acid terminated, |
| | M_{w} 76,000-115,000 |
| RG 858S, Poly(D,L-Lactide-co-Glycolide) | lactide:glycolide 85:15, ester |
| | terminated, M_{w} 190,000-240,000 |
| RG 858H, Poly(D,L-Lactide-co-Glycolide) | lactide:glycolide 85:15, acid terminated, |
| | M_{w} 190,000-240,000 |

TABLE 3
Description of certain PLA or PLGA polymers herein.

| | Inherent | End | |
|---|---|---|---|
| | Viscosity | Group | Molar Ratio |
| DL 03 A, Poly(D,L-Lactide) | 0.2-0.4 dL/g | Acid | lactide:glycolide |
| | | | 100:0 |
| DL 03 E, Poly(D,L-Lactide) | 0.2-0.4 dL/g | Ester | lactide:glycolide |
| | | | 100:0 |
| DLG 8505 A, Poly(D,L- | 0.4-0.6 dL/g | Acid | lactide:glycolide |
| Lactide-co-Glycolide) | | | 85:15 |
| DLG 8505 E, Poly(D,L- | 0.4-0.6 dL/g | Ester | lactide:glycolide |
| Lactide-co-Glycolide) | | | 85:15 |
| DLG 8503 A, Poly(D,L- | 0.2-0.4 dL/g | Acid | lactide:glycolide |
| Lactide-co-Glycolide) | | | 85:15 |
| DLG 8503 E, Poly(D,L- | 0.2-0.4 dL/g | Ester | lactide:glycolide |
| Lactide-co-Glycolide) | | | 85:15 |
| DLG 7505 A, Poly(D,L- | 0.4-0.6 dL/g | Acid | lactide:glycolide |
| Lactide-co-Glycolide) | | | 75:25 |
| DLG 7505 E, Poly(D,L- | 0.4-0.6 dL/g | Ester | lactide:glycolide |
| Lactide-co-Glycolide) | | | 75:25 |
| DLG 7503 A, Poly(D, L- | 0.2-0.4 dL/g | Acid | lactide:glycolide |
| Lactide-co-Glycolide) | | | 75:25 |
| DLG 7503 E, Poly(D,L- | 0.2-0.4 dL/g | Ester | lactide:glycolide |
| Lactide-co-Glycolide) | | | 75:25 |
| DLG 6505 A, Poly(D,L- | 0.4-0.6 dL/g | Acid | lactide:glycolide |
| Lactide-co-Glycolide) | | | 65:35 |
| DLG 6505 E, Poly(D,L- | 0.4-0.6 dL/g | Ester | lactide:glycolide |
| Lactide-co-Glycolide) | | | 65:35 |
| DLG 6503 A, Poly(D,L- | 0.2-0.4 dL/g | Acid | lactide:glycolide |
| Lactide-co-Glycolide) | | | 65:35 |
| DLG 6503 E, Poly(D,L- | 0.2-0.4 dL/g | Ester | lactide:glycolide |
| Lactide-co-Glycolide) | | | 65:35 |
| DLG 5005 A, Poly(D,L- | 0.4-0.6 dL/g | Acid | lactide:glycolide |
| Lactide-co-Glycolide) | | | 50:50 |
| DLG 5005 E, Poly(D,L- | 0.4-0.6 dL/g | Ester | lactide:glycolide |
| Lactide-co-Glycolide) | | | 50:50 |
| DLG 5003 E, Poly(D,L- | 0.2-0.4 dL/g | Acid | lactide:glycolide |
| Lactide-co-Glycolide) | | | 50:50 |
| DLG 5003 E, Poly(D,L- | 0.2-0.4 dL/g | Ester | lactide:glycolide |
| Lactide-co-Glycolide) | | | 50:50 |

TABLE 4
Descriptions of certain polymer compositions herein.

| | Polymer 1 | | Polymer 2 | | Implant |
|---|---|---|---|---|---|
| Composition # | Name | % | Name | % | Needle Size |
| 1 | PLGA | 100 | — | — | 23G |
| | 6503E | | | | |
| 2 | PLGA | 50 | PLGA | 50 | |
| | 6503E | | 6503A | | |
| 3 | PLGA | 90 | PEA X25 | 10 | |
| | 6503E | | | | |
| 4 | PLGA | 80 | PEA X25 | 20 | |
| | 6503E | | | | |
| 5 | PLGA | 70 | PEA X25 | 30 | |
| | 6503E | | | | |
| 6 | PLGA | 70 | PLGA | 30 | |
| | 6503E | | 5005A | | |
| 7 | PLGA | 50 | PLGA | 50 | |
| | 6503E | | 5005A | | |
| 8 | PLGA | 30 | PLGA | 70 | |
| | 6503E | | 5005A | | |
| 9 | PLGA | 50 | PLGA | 50 | |
| | 6503A | | 5005E | | |
| 10 | PLGA | 100 | — | — | |
| | 7505E | | | | |
| 11 | PLGA | 50 | PLGA | 50 | |
| | 7505E | | 7505A | | |
| 12 | PLGA | 90 | PLGA | 10 | |
| | 7505E | | 5005A | | |
| 13 | PLGA | 70 | PLGA | 30 | |
| | 7505E | | 5005A | | |
| 14 | PLGA | 50 | PLGA | 50 | |
| | 7505E | | 5005A | | |
| 15 | PLGA | 90 | PLGA | 10 | |
| | 7505E | | 6503A | | |
| 16 | PLGA | 70 | PLGA | 30 | |
| | 7505E | | 6503A | | |
| 17 | PLGA | 50 | PLGA | 50 | |
| | 7505E | | 6503A | | |
| 18 | PLGA | 90 | PEA X25 | 10 | |
| | 7505E | | | | |
| 19 | PLGA | 80 | PEA X25 | 20 | |
| | 7505E | | | | |
| 20 | PLGA | 70 | PEA X25 | 30 | |
| | 7505E | | | | |
| 21 | PLGA | 100 | — | — | |
| | 8503E | | | | |
| 22 | PLGA | 50 | PLGA | 50 | |
| | 8503E | | 8503A | | |
| 23 | PLGA | 90 | PLGA | 10 | |
| | 8503E | | 5005A | | |
| 24 | PLGA | 70 | PLGA | 30 | |
| | 8503E | | 5005A | | |
| 25 | PLGA | 50 | PLGA | 50 | |
| | 8503E | | 5005A | | |
| 26 | PLGA | 90 | PEA X25 | 10 | |
| | 8503E | | | | |
| 27 | PLGA | 80 | PEA X25 | 20 | |
| | 8503E | | | | |
| 28 | PLGA | 70 | PEA X25 | 30 | |
| | 8503E | | | | |
| 29 | PLGA | 70 | PLGA | 30 | |
| | 8503E | | 6503A | | |
| 30 | PLGA | 50 | PLGA | 50 | |
| | 8503E | | 6503A | | |
| 31 | PLGA | 30 | PLGA | 70 | |
| | 8503E | | 6503A | | |
| 32 | PLA DL 03 | 100 | — | — | |
| | E | | | | |
| 33 | PLA DL 03 | 100 | — | — | |
| | A | | | | |
| 34 | PLA DL 03 | 50 | PLGA | 50 | |
| | E | | 5005A | | |
| 35 | PLGA | 70 | PLGA | 30 | 25G |
| | 7505E | | 5005A | | |

In a preferred embodiment, the polymer matrix comprises 100% DLG 7505E. Another preferred embodiment comprises 70% DLG 7505E and 30% DLG 5005A. In another preferred embodiment, the polymer matrix comprises 90% DLG 7505E and 10% PEA X25.

The implants described herein, comprising an API and a polymer matrix, may have any suitable weight. In some embodiments, the weight of the implant may be about 300 mg to about 1200 mg, about 700-1100 mg, about 800-1000 mg, about 300-400 mg, about 400-500 mg, about 500-600 mg, about 600-700 mg, about 800-900 mg, about 900-1000 mg, about 1000-1100 mg, about 1100-1200 mg, or about 425 mg, about 450 mg, about 475 mg, about 825 mg, about 850 mg, about 875 mg, about 925 mg, about 950 mg, about 975 mg, or any weight in a range bounded by any of these values.

### Intravitreal Delivery and Methods

Topical administration of API's generally achieves low concentration in the vitreous and is not commonly used for the treatment of retinal diseases. Systemic treatment is also limited, as the blood-retina-barrier limits the ability of the API to reach the eye, and high doses may cause systemic adverse effects. Periocular (subconjunctival, sub-Tenon, or retrobulbar) injections can bypass the blood-retina-barrier without intraocular penetration. However, the injected API still needs to traverse the sclera, which is less permeable to larger molecules, and may not achieve therapeutic drug levels at the level of the retina. Therefore, alternate routes of delivery are needed.

The compositions of the present disclosure, comprising a polymer matrix and a PKal inhibitor with or without an anti-VEGF compound, make use of intravitreal delivery for the treatment of retinal disease, including DME. In some embodiments, the polymers and the API(s) are powdered, blended together, and heated at up to about 180 °C to form a blended polymer matrix. The blended polymer matrix is rolled to an appropriate thickness and then implants are formed with a tooling sheet or may be melt extruded. The compositions of the present disclosure may utilize any suitable drug-polymer implant manufacture technology to prepare an implant to be administered via an applicator syringe needle. The implants described herein are homogenous blends of the polymer component(s) and the active agent(s). The implants of the present disclosure are solid structures (e.g., not gels or liquids or powders), and are stable for long periods of storage under ambient conditions (e.g., longer than at least 2 years).

Injections of the implants described herein into the vitreous cavity of the subjects suffering from DME or other retinal vascular disease are performed using suitable small-gauge needles. Some embodiments include needles having a size between about 23 gauge (23G) and about 30G. In some embodiments, a 25G needle is used for injection. In other embodiments, a 23G needle is used for injection. Some embodiments include the use of 30G needles for injection. In some embodiments, the needles are loaded with the implant prepared above and administered in the IVT of the subject. In some embodiments, the administration of the implant is performed surgically, e.g., where the patient is under general anesthesia or local anesthesia (e.g., retrobulbar block). In some embodiments, the implant delivers therapeutically effective levels of API to the structures of the back of the eye, including the retina, retinal pigment epithelium/choroid, optic nerve head, and vitreous humor.

In some embodiments, more than one implant may be injected intravitreally to the subject. In some embodiments, one or more implants comprising a PKal inhibitor (e.g., avoralstat or a pharmaceutically acceptable salt thereof), may be administered to a patient intravitreally. In other embodiments, one or more implants comprising a VEGF inhibitor (e.g., axitinib) may be administered intravitreally to a patient. Other embodiments include injecting a single implant comprising both the PKal inhibitor (e.g., avoralstat or a pharmaceutically acceptable salt thereof) and the VEGF inhibitor (e.g., axitinib) into the vitreous of the subject.

In some embodiments, two or more implants are administered in separate injector needles, in separate injections (e.g., a first implant in a first injector needle and a second implant in a second injector needle, the first implant and the second implant being optionally different in composition, e.g., the first implant being an implant composition described herein comprising a PKal inhibitor (e.g., avoralstat or a pharmaceutically acceptable salt thereof) and the second implant being an implant composition described herein comprising a VEGF inhibitor (e.g., axitinib)). Some embodiments include administering more than one implant using a single injector needle in a single injection (e.g., a first implant and a second implant, the first implant and the second implant being the same or optionally different in composition, e.g., the first implant (or first and second implant) being an implant composition described herein comprising a PKal inhibitor (e.g., avoralstat or a pharmaceutically acceptable salt thereof) and the second implant being an implant composition described herein comprising a VEGF inhibitor (e.g., axitinib)). In some embodiments, two implants may be administered in one injection using a single injector needle. In other embodiments, three implants may be administered in a single injection from a single injector needle. In some embodiments, four implants may be administered in a single injection using a single injector needle. In embodiments where two or more implants are administered in a single injection using the same injector needle, the implants may comprise at least one PKal inhibitor (e.g., avoralstat or a pharmaceutically acceptable salt thereof) implant and at least one VEGF inhibitor (e.g., axitinib) implant. In some embodiments, one implant comprising a PKal inhibitor (e.g., avoralstat or a pharmaceutically acceptable salt thereof) and one separate implant comprising a VEGF inhibitor (e.g., axitinib) are administered together in one injection using the same injector needle. In embodiments where three or more implants are administered by a single injection using a single injector needle, the implants may comprise at least one PKal inhibitor (e.g., avoralstat or a pharmaceutically acceptable salt thereof) implant and at least one VEGF inhibitor (e.g., axitinib) implant. In embodiments where four implants are administered using a single injector needle in a single injection, the implants may comprise at least one PKal inhibitor (e.g., avoralstat or a pharmaceutically acceptable salt thereof) implant and at least one VEGF inhibitor (e.g., axitinib) implant.

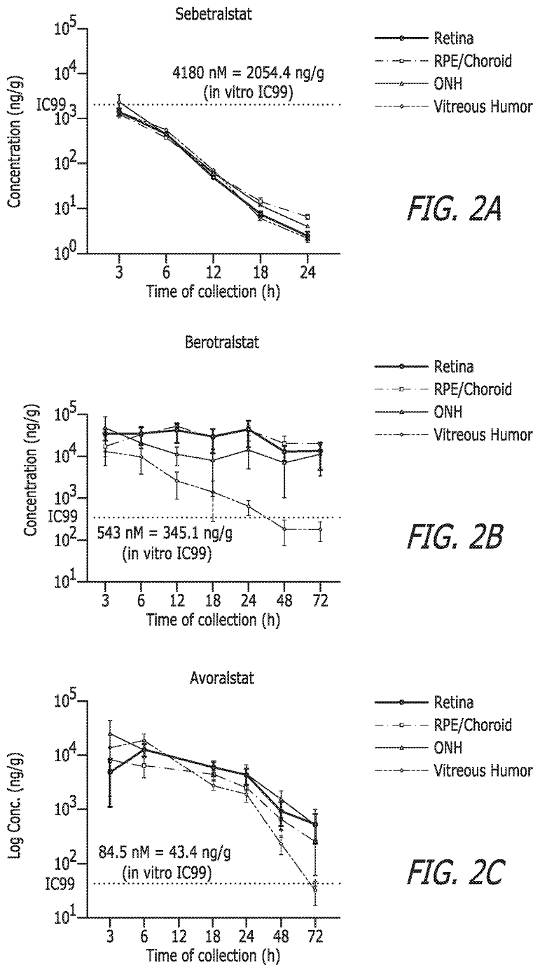

The preparation of the API/polymer matrix implant comprises a heating step and an implant preparation step, as detailed above. In some embodiments, avoralstat (see FIG. 1A and FIG. 1B) and berotralstat (see FIG. 1C and FIG. 1D) polymer matrices survive the thermal melt/implant formation procedure as determined by visual inspection, while the sebetralstat (see FIG. 1E and FIG. 1F) polymer matrix deliquesces under the same conditions (see Example 1). In another embodiment, berotralstat undergoes significant degradation when the berotralstat/polymer matrix is subjected to implant processing temperatures, while the implants containing avoralstat show little if any degradation of the API, as determined by HPLC analysis (see Example 1). In another example, berotralstat implants have an ocular tissue clearance rate that is too slow to maintain a therapeutically effective concentration of the API in the tissues of the eye, and avoid accumulation of the API, for the desired duration of implant residence (see Example 2 and FIG. 2B). In another example, sebetralstat has an ocular duration that is too short to achieve a therapeutically effective concentration of the API in the tissues of the eye for the desired duration of implant residence (see Example 2 and FIG. 2A). It has been observed that the implants comprising avoralstat achieve a therapeutically effective concentration of avoralstat in ocular tissues over an acceptable duration of implant residence (see Example 2 and FIG. 2C).

In vivo studies to determine target ocular tissue pharmacokinetic (PK) profiles of avoralstat implants were conducted in dutch belted rabbits. Concentrations of avoralstat in ocular tissue (iris ciliary body (ICB), retina, retinal pigment epithelium (RPE)/choroid, optic nerve head (ONH), and vitreous humor) were measured at specific time points (1 month, 2 months, 3 months, 4.5 and 6 months) after an intravitreal injection of an implant comprising avoralstat and a polymer matrix. Samples were processed and analyzed for avoralstat acid using a qualified LC-MS/MS analytical method.

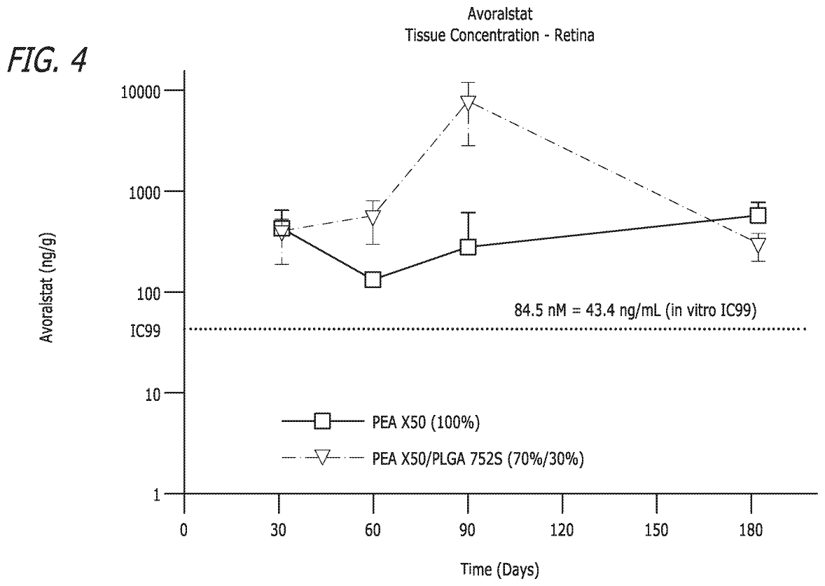

A first study is described in Example 3 and results are shown in FIG. 4. The implant compositions used in the first study comprised: 1) 60% w/w PEA III X50 and 40% w/w avoralstat; and 2) 42% w/w PEA III X50, 18% w/w PLGA 752S, and 40% w/w avoralstat. The tissue concentration of avoralstat in the retina was above the IC₉₉ at all time points for both formulations. It was determined that avoralstat was released from the implant to the target tissue at an acceptable rate.

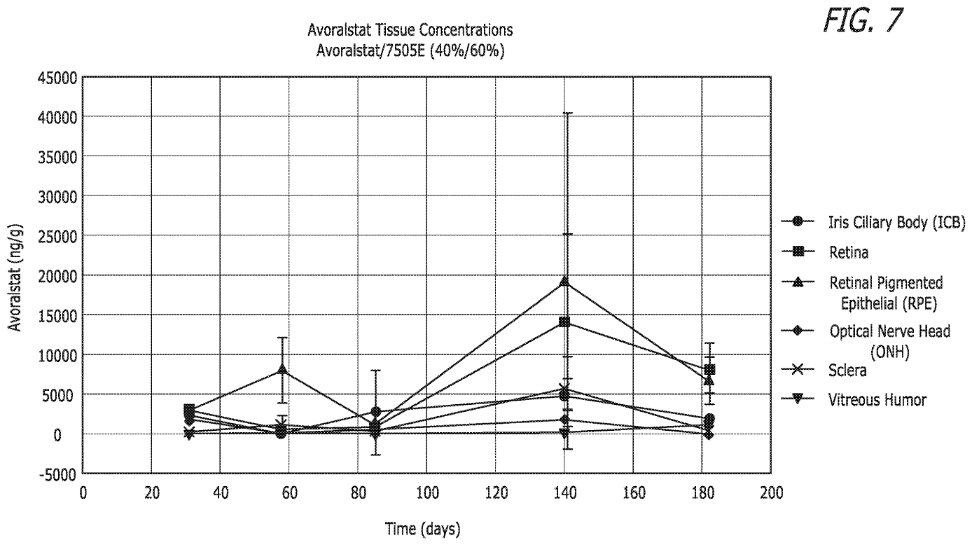

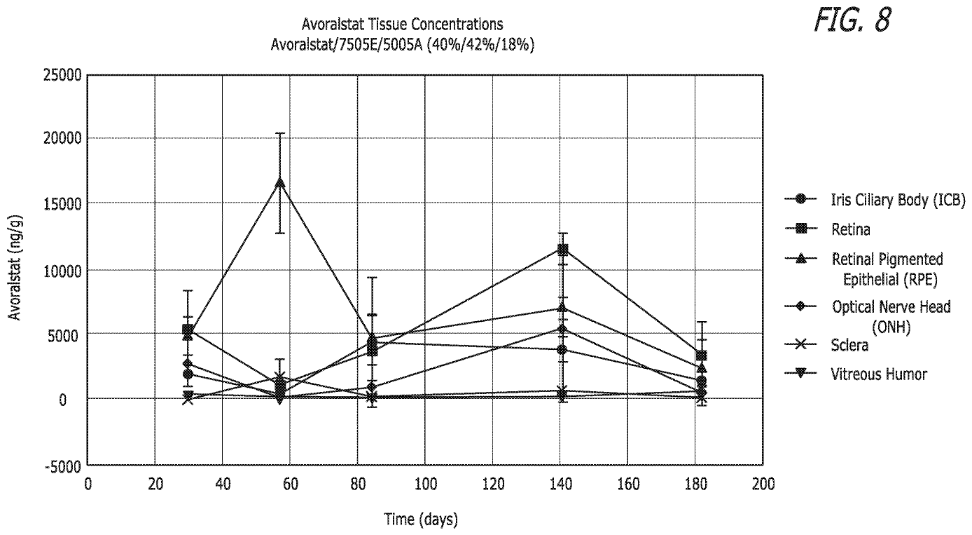

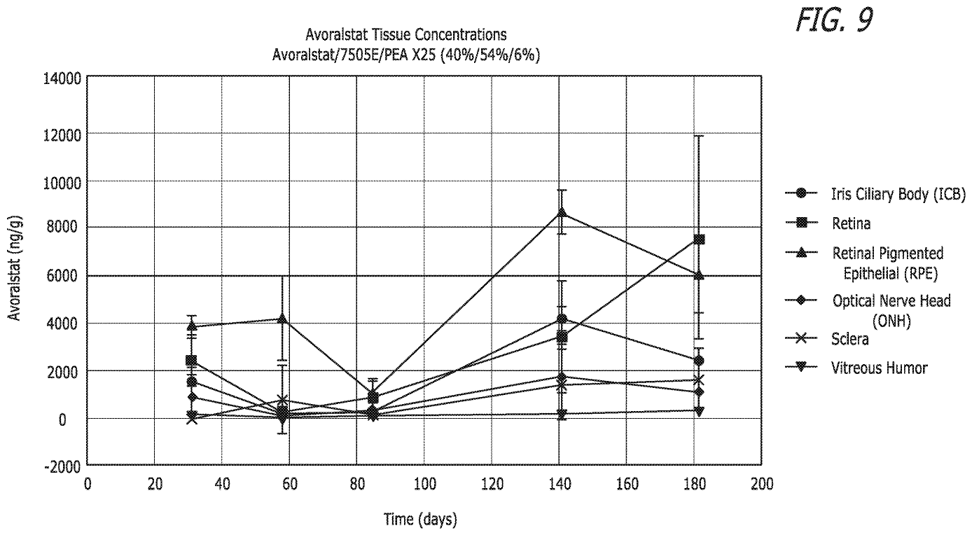

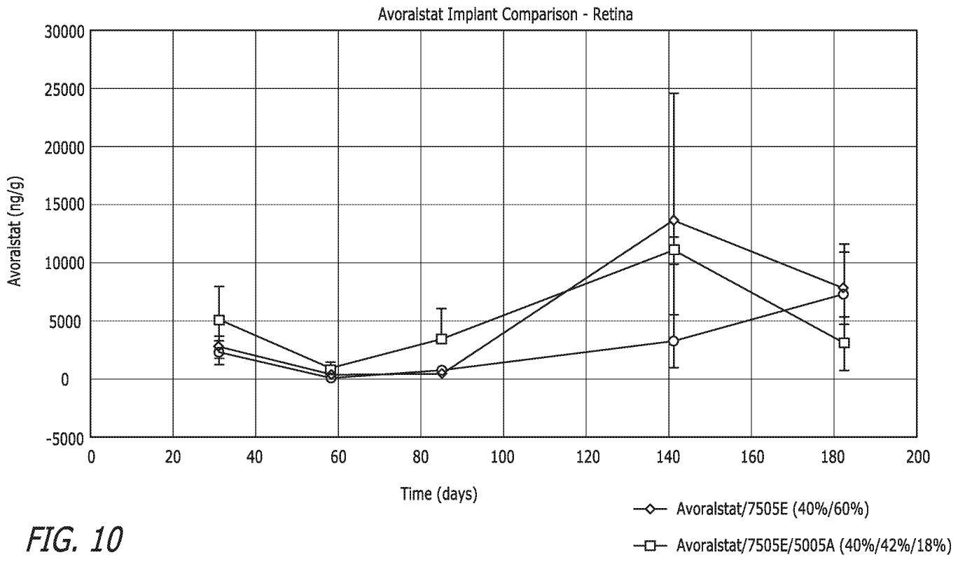

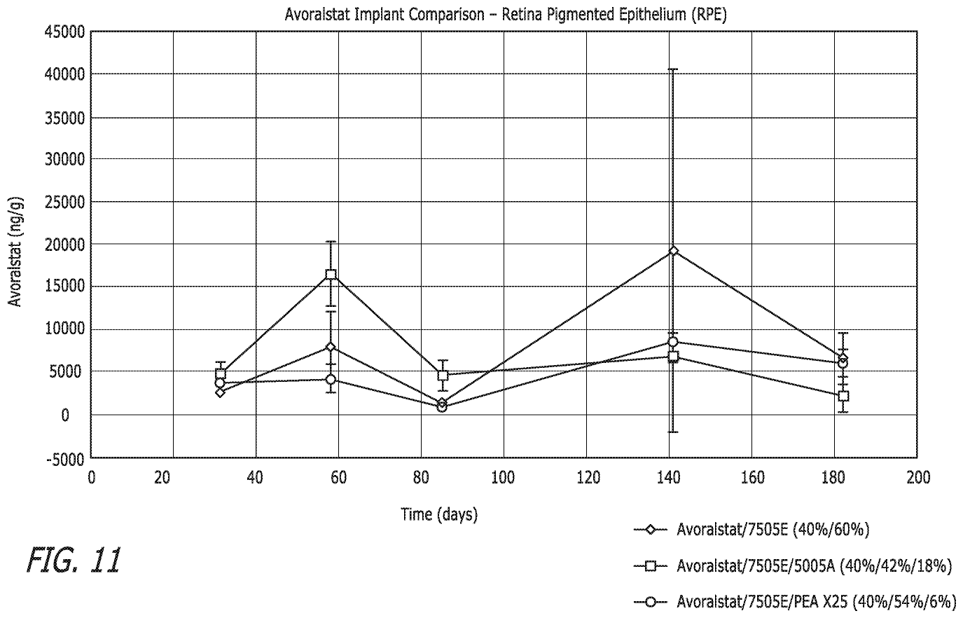

The implant compositions used in another study (see Example 16) comprised three groups: 1A) 60% w/w PLGA 7505E and 40% w/w avoralstat; 2A) 42% w/w PLGA 7505E, 18% w/w PLGA 5005A, and 40% w/w avoralstat; 3A) 54% w/w PLGA 7505E, 6% w/w PEA III X25, and 40% w/w avoralstat. The results of the study are shown in FIGS. 7-11: FIG. 7 shows the results for group 1A; FIG. 8 shows the results for group 2A; and FIG. 9 shows the results for group 3A. Each of the groups succeeded in delivering concentrations of avoralstat to each of the ocular tissues. It was determined that the implants of group 2A), comprising 42% w/w PLGA 7505E, 18% w/w PLGA 5005A, and 40% w/w avoralstat, exhibited a consistently higher release of avoralstat as compared to the other two groups. The retinal and RPE/choroid concentrations of avoralstat (see FIG. 10 and FIG. 11, respectively) delivered from each of the implants was above the IC₉₉ at all timepoints tested. Each group delivered avoralstat to the target tissues at an acceptable rate.

It has been determined herein that the implants provide the highest concentrations of avoralstat in the RPE. The tissue distribution of avoralstat in these in vivo studies, from highest to lowest concentrations, is RPE>retina>ONH>ICB>sclera>vitreous humor. Without being bound by theory, this gradient may suggest that preferential accumulation of avoralstat is found in pigmented or metabolically active tissues.

Thus, in some embodiments, provided herein are methods of treating DME and other retinal vascular diseases in a subject in need thereof, comprising administering to an eye of the subject an ocular implant formed of a polymer matrix described herein and a PKal inhibitor (e.g., avoralstat or a pharmaceutically acceptable salt thereof), optionally including one or more API (e.g., VEGF inhibitor or VEGFR inhibitor). In some embodiments, the administration is by intravitreal injection to an eye of the subject. In some embodiments, the administration is by surgical administration, optionally including a step of maintaining the subject under general or local anesthesia during the administration of the polymer matrix (e.g., an IVT implant) to the subject.

In some embodiments of the methods herein, a subject, suffering from DME and treated with the intravitreal implants of the present disclosure, experiences an improvement in measurement of a sign or symptom of diabetic macular edema as compared to administration of an anti-VEGF therapeutic to the subject.

In some embodiments of the methods herein, a subject, suffering from DME and treated with the intravitreal implants of the present disclosure, experiences an improvement in measurement of a sign or symptom of diabetic macular edema as compared to administration of dexamethasone or fluocinolone acetonide to the subject.

In some embodiments of the methods herein, a subject, suffering from DME and treated with the intravitreal implants of the present disclosure, experiences an at least 10% improvement in measurement of a sign or symptom of diabetic macular edema as compared to administration, by IVT implant, of 0.7 mg dexamethasone in a polymer matrix (OZURDEX®) or 0.19 mg fluocinolone acetonide in a drug delivery system (ILUVIEN®). In some embodiments, the subject suffering from DME and treated with the intravitreal implants of the present disclosure, experiences an at least 10% improvement in measurement of a sign or symptom of diabetic macular edema as compared to administration of OZURDEX® or ILUVIEN® after at least 1 month, at least 2 months, at least 3 months, at least 4 months, at least 5 months, at least 6 months, at least 7 months, at least 8 months, at least 9 months, at least 10 months, at least 1 months, or at least 1 year after administration.

In addition to the release of the API from the implant, another consideration is the complete dissipation (e.g., erosion) of the polymer matrix. As described herein, the intravitreal implant compositions herein comprise one or more than one API and a polymer matrix. Dissipation rates of polymer that correspond directly with the rate of API release may be achieved in some embodiments of the present disclosure. In some intravitreal implant compositions, the polymer matrix component may not be fully eroded even though the one or more API has been completely released from the implant over a certain period of time following implantation at a target site. The duration or presence of such a remnant polymer matrix, in some embodiments, may be minimized or eliminated by bioerosion. In some embodiments of the methods herein, the complete dissipation or bioerosion of the polymer from the back of the eye (e.g., retinal tissue) takes place in less than 12 months, e.g., from initial implantation. In some embodiments of the methods herein, the dissipation of polymer (e.g., no detectable polymer concentration or particle size) is achieved over about 2 months to about 12 months, about 2-4 months, about 4-6 months, about 6-8 months, about 8-10 months, about 10-12 months, about 3-6 months, about 6-9 months, about 6-12 months, about 9-12 months, or about 3 months, about 4 months, about 5 months, about 6 months, about 7 months, about 8 months, about 9 months, about 10 months, about 11 months, about 12 months, about 12-16 months or any time period from initial implantation bounded by these ranges. In some embodiments of the methods herein, the remnant polymer matrix is eroded completely (e.g., to undetectable concentration or particle size) within about 0, 1, 2, 3, 4, 5, 6, 9, or 12 months of implantation of the intravitreal implant composition.

In some embodiments of the methods herein, the complete release of the API into vitreous humor and the tissues of the back of the eye (e.g., retinal tissue) takes place over about 2 months to about 12 months, about 2-4 months, about 4-6 months, about 6-8 months, about 8-10 months, about 10-12 months, about 3-6 months, about 6-9 months, about 6-12 months, about 9-12 months, or about 3 months, about 4 months, about 5 months, about 6 months, about 7 months, about 8 months, about 9 months, about 10 months, about 11 months, about 12 months, or any time period bounded by a range from any pair of these values. In some embodiments of the methods herein, the complete release of the API into vitreous humor and the tissues of the back of the eye (e.g., retinal tissue) takes place over a period of about 6 months or a period from about 6 months up to about 12 months. In some embodiments of the methods herein, the API is released from the implant at a therapeutically relevant amount (e.g., above an IC₉₉ value, e.g., above about 80-90 nM (e.g., about 83-87, about 84, about 84.5, about 85, or about 86 nM) for avoralstat or a pharmaceutically acceptable salt thereof) on an average daily basis for at least half a year (e.g., about 6 months) or for at least half a year and up to one year (e.g., about 6-12 months).

In some embodiments of the methods herein, the composition of the intravitreal implant composition comprises:
A) 60% w/w PEA III X50 and 40% w/w PKal inhibitor (e.g., avoralstat or a pharmaceutically acceptable salt thereof);
B) 42% w/w PEA III X50, 18% w/w PLGA 752S, and 40% w/w PKal inhibitor (e.g., avoralstat or a pharmaceutically acceptable salt thereof);
C) 30% w/w PLGA 503, 30% PLGA 503H, and 40% w/w PKal inhibitor (e.g., avoralstat or a pharmaceutically acceptable salt thereof);
D) 60% w/w PEA III X25 and 40% w/w PKal inhibitor (e.g., avoralstat or a pharmaceutically acceptable salt thereof);
E) 42% w/w PEA III X25, 18% w/w PLGA 752S, and 40% w/w PKal inhibitor (e.g., avoralstat or a pharmaceutically acceptable salt thereof);
F) 60% w/w PEA III X75 and 40% w/w PKal inhibitor (e.g., avoralstat or a pharmaceutically acceptable salt thereof);
G) 60% w/w PLGA 7505E and 40% w/w PKal inhibitor (e.g., avoralstat or a pharmaceutically acceptable salt thereof);
H) 42% w/w PLGA 7505E, 18% w/w PLGA 5005A, and 40% w/w PKal inhibitor (e.g., avoralstat or a pharmaceutically acceptable salt thereof); or
I) 54% w/w PLGA 7505E, 6% w/w PEA III X25, and 40% w/w PKal inhibitor (e.g., avoralstat or a pharmaceutically acceptable salt thereof).

### EMBODIMENTS

Embodiment 1. A composition, comprising a polymeric matrix and a plasma kallikrein (PKal) inhibitor, wherein the PKal inhibitor is dispersed within the polymeric matrix, and the polymeric matrix is a bioerodible polymeric ocular intravitreal implant.

Embodiment 2. The composition of embodiment 1, wherein the PKal inhibitor is berotralstat or a pharmaceutically acceptable salt thereof.

Embodiment 3. The composition of embodiment 1, wherein the PKal inhibitor is sebetralstat or a pharmaceutically acceptable salt thereof.

Embodiment 4. The composition of embodiment 1, wherein the PKal inhibitor is avoralstat or a pharmaceutically acceptable salt thereof.

Embodiment 5. The composition of embodiment 1, wherein the composition further comprises at least one compound selected, independently, from a vascular endothelial growth factor (VEGF) inhibitor or a vascular endothelial growth factor receptor (VEGFR) inhibitor.

Embodiment 6. The composition of embodiment 1, wherein the composition further comprises one or more of axitinib, cabozantinib, fruquintinib, lenvatinib, pazopanib, ponatinib, regorafenib, sorafenib, sunitinib, or tivozanib, or a pharmaceutically acceptable salt thereof.

Embodiment 7. The composition of embodiment 6, wherein the axitinib, cabozantinib, fruquintinib, lenvatinib, pazopanib, ponatinib, regorafenib, sorafenib, sunitinib, or tivozanib, or a pharmaceutically acceptable salt thereof, is dispersed within the polymeric particle.

Embodiment 8. The composition of embodiment 1, wherein the composition further comprises one or more of aflibercept, bevacizumab, brolucizumab, faricimab, or ranibizumab, or a biosimilar thereof.

Embodiment 9. The composition of embodiment 1, wherein the polymeric matrix comprises a polyesteramide polymer or a poly(lactic-co-glycolic) acid polymer.

Embodiment 10. The composition of embodiment 1, wherein the polymeric matrix comprises a polyesteramide polymer and a poly(lactic-co-glycolic) acid polymer.

Embodiment 11. The composition of embodiment 9 or 10, wherein the polyesteramide polymer comprises an α-amino acid-diol-diester polyesteramide, wherein the α-amino acid-diol-diester polyesteramide comprises PEA III X25, PEA III X50, PEA III X65, or PEA III X75 (e.g., PEA III X25 or PEA III X50).

Embodiment 12. A method of treating diabetic macular edema in a subject in need thereof, comprising intravitreal administration of the composition of one of embodiments 1-11 to a subject in need thereof.

Embodiment 13. The method of embodiment 12, wherein the subject experiences an at least 10% improvement in measurement of a sign or symptom of diabetic macular edema as compared to administration of dexamethasone or fluocinolone acetonide to the subject.

Embodiment 14. The method of embodiment 12 or 13, wherein the implant releases the PKal inhibitor at an effective concentration over at least three months.

Embodiment 15. The method of embodiment 12 or 13, wherein the implant releases the PKal inhibitor at an effective concentration over at least six months.

Embodiment 16. The method of embodiment 12 or 13, wherein the implant releases the PKal inhibitor and the vascular endothelial growth factor (VEGF) inhibitor or the vascular endothelial growth factor receptor (VEGFR) at an effective concentration over at least three months.

Embodiment 17. An intravitreal implant, comprising a bioerodible polymeric matrix and a plasma kallikrein (PKal) inhibitor selected from avoralstat or a pharmaceutically acceptable salt thereof, wherein the PKal inhibitor is dispersed within the bioerodible polymeric matrix.

Embodiment 18. The intravitreal implant of embodiment 17, wherein the PKal inhibitor is avoralstat.

Embodiment 19. The intravitreal implant of embodiment 17, wherein the PKal inhibitor is a pharmaceutically acceptable salt of avoralstat.

Embodiment 20. The intravitreal implant of embodiment 17, wherein the PKal inhibitor is a solid form of avoralstat or a pharmaceutically acceptable salt thereof.

Embodiment 21. The intravitreal implant of one of embodiments 17-20, comprising:
A) 60% w/w PEA III X50 and 40% w/w PKal inhibitor;
B) 42% w/w PEA III X50, 18% w/w PLGA 752S, and 40% w/w PKal inhibitor;
C) 30% w/w PLGA 503, 30% PLGA 503H, and 40% w/w PKal inhibitor;
D) 60% w/w PEA III X25 and 40% w/w PKal inhibitor;
E) 42% w/w PEA III X25, 18% w/w PLGA 752S, and 40% w/w PKal inhibitor;
F) 60% w/w PEA III X75 and 40% w/w PKal inhibitor;
G) 60% w/w PLGA 7505E and 40% w/w PKal inhibitor;
H) 42% w/w PLGA 7505E, 18% w/w PLGA 5005A, and 40% w/w PKal; or
I) 54% w/w PLGA 7505E, 6% w/w PEA III X25, and 40% w/w PKal inhibitor.

Embodiment 22. The intravitreal implant of one of embodiments 17-21, further comprising at least one compound selected, independently, from a vascular endothelial growth factor (VEGF) inhibitor or a vascular endothelial growth factor receptor (VEGFR) inhibitor.

Embodiment 23. The intravitreal implant of one of embodiments 17-21, further comprising one or more of axitinib, cabozantinib, fruquintinib, lenvatinib, pazopanib, ponatinib, regorafenib, sorafenib, sunitinib, or tivozanib, or a pharmaceutically acceptable salt thereof.

Embodiment 24. The intravitreal implant of one of embodiments 17-21, further comprising axitinib or a pharmaceutically acceptable salt thereof.

Embodiment 25. The intravitreal implant of one of embodiments 22-24, wherein the VEGF inhibitor, VEGFR inhibitor, axitinib, cabozantinib, fruquintinib, lenvatinib, pazopanib, ponatinib, regorafenib, sorafenib, sunitinib, or tivozanib, or a pharmaceutically acceptable salt thereof, is dispersed within the bioerodible polymeric matrix.

Embodiment 26. The intravitreal implant of one of embodiments 17-21, further comprising one or more of aflibercept, bevacizumab, brolucizumab, faricimab, or ranibizumab, or a biosimilar thereof.

Embodiment 27. The intravitreal implant of one of embodiments 17-26, wherein the bioerodible polymeric matrix comprises a polyesteramide polymer, a poly(lactic-co-glycolic) acid polymer, or a polylactic acid polymer.

Embodiment 28. The intravitreal implant of one of embodiments 17-26, wherein the bioerodible polymeric matrix comprises a polyesteramide polymer and a poly(lactic-co-glycolic) acid polymer.

Embodiment 29. The intravitreal implant of embodiment 28, wherein the poly(lactic-co-glycolic) acid polymer comprises a mole fraction of lactic acid and a mole fraction of glycolic acid, wherein the mole fraction of lactic acid is not less than the mole fraction of glycolic acid.

Embodiment 30. The intravitreal implant of one of embodiments 17-29, wherein the polyesteramide polymer comprises an α-amino acid-diol-diester polyesteramide, wherein the α-amino acid-diol-diester polyesteramide comprises PEA III X25, PEA III X35, PEA III X50, PEA III X65, or PEA III X75, or a mixture thereof.

Embodiment 31. The intravitreal implant of one of embodiments 17-30, wherein the intravitreal implant is configured to release a therapeutically effective amount of the PKal inhibitor, wherein the therapeutically effective amount is, on a daily basis, an average of less than about 0.5-0.6% of the total molar amount of the PKal inhibitor.

Embodiment 32. An intravitreal implant, which is a combination comprising an intravitreal implant comprising axitinib or a pharmaceutically acceptable salt thereof and an intravitreal implant comprising the intravitreal implant of one of claims 17-31.

Embodiment 33. A method of treating diabetic macular edema in a subject in need thereof, comprising intravitreal administration of the intravitreal implant of one of embodiments 17-32 to the subject.

Embodiment 34. A method of treating diabetic retinopathy, proliferative diabetic retinopathy, macular edema (e.g., diabetic macular edema), age-related macular degeneration, or retinal vein occlusion in a subject in need thereof, comprising intravitreal administration of avoralstat or a pharmaceutically acceptable salt thereof and intravitreal administration of axitinib or a pharmaceutically acceptable salt thereof to the subject.

Embodiment 35. A method of treating diabetic retinopathy, proliferative diabetic retinopathy, macular edema (e.g., diabetic macular edema), age-related macular degeneration, or retinal vein occlusion in a subject in need thereof, comprising intravitreal administration of the intravitreal implant of embodiment 32 to the subject.

Embodiment 36. The method of one of embodiments 33-35, wherein the subject experiences an at least 10% improvement in measurement of a sign or symptom of diabetic macular edema as compared to administration by IVT implant of 0.7 mg dexamethasone or 0.19 mg fluocinolone acetonide to the subject.

Embodiment 37. The method of one of embodiments 33-36, wherein the intravitreal implant releases a therapeutically effective amount of the PKal inhibitor for at least six months, wherein the therapeutically effective amount is, on a daily basis, an average of less than about 0.5-0.6% of the total molar amount of the PKal inhibitor.

Embodiment 38. The method of one of embodiments 33-37, wherein the intravitreal implant releases a therapeutically effective amount of the vascular endothelial growth factor (VEGF) inhibitor or the vascular endothelial growth factor receptor (VEGFR) inhibitor for at least three months, wherein the therapeutically effective amount is, on a daily basis, an average of less than about 1.0-1.2% of the total molar amount of the VEGF inhibitor or the VEGFR inhibitor.

Embodiment 39. The method or the intravitreal implant of any of the previous embodiments, wherein the intravitreal implant is prepared by a process comprising a thermal melt step.

### EXAMPLES

The following examples further illustrate embodiments of the present disclosure. However, they are in no way a limitation of the teachings or disclosure as described herein.

### Example 1

A polymer matrix comprising avoralstat, berotralstat, or sebetralstat is prepared by powdering and dispensing the solid polymer PEA III X50, dispensing berotralstat powder, and blending them together on a hot plate at 180 °C. A roller then makes a film on the 180 °C hot plate. The resulting film is then laminated at 170 °C with a tooling sheet to form implants sized for administration using a pharmaceutical syringe needle. The composition of the implant is 60% w/w of PEA III X50 and 40% PKal inhibitor avoralstat, berotralstat, or sebetralstat. After cooling, implants were picked and API stability was assessed by visual inspection and HPLC. Films and corresponding implants are shown in FIG. 1A, FIG. 1B, FIG. 1C, FIG. 1D, FIG. 1E, and FIG. 1F. It was observed that homogeneous films and good quality implants were made using avoralstat. On the other hand, berotralstat and sebetralstat resulted in inconsistent films and poor quality implants. Moreover, from implant HPLC analysis, it was observed that both avoralstat and sebetralstat implants kept their chemical purity post manufacturing, while berotralstat resulted in further degradation. Sebetralstat, despite keeping its chemical purity, melted during manufacturing. Overall, avoralstat is the only one that kept its physical form and remained chemically stable during manufacturing making it the only one amenable to thermal melt implant manufacturing.

### Example 2

A study to determine target ocular tissue PK profiles of PKal inhibitors was conducted in rabbits. Concentrations of avoralstat, berotralstat, or sebetralstat in ocular tissues (retina, retinal pigment epithelium/choroid, optic nerve head, and vitreous humor) were measured at specific time points (3h, 6 h, 12 h 18 h, 24 h, 48h, and 72h) after a 50 μL bolus intravitreal injection of a given PKal inhibitor at 0.5 mg/ml (5% PKal inhibitor/0.8% Myrl S40/propylene glycol in 20 mM Citrate buffer). The results of the study are shown in FIG. 2A, FIG. 2B, and FIG. 2C. It was determined that sebetralstat was released very quickly from ocular tissues, berotralstat was released too slowly from ocular tissues, and avoralstat was released from the implant to the target tissues at an acceptable rate.

### Example 3

A study to determine target ocular tissue pharmacokinetic (PK) profiles of PKal inhibitors was conducted in rabbits. Concentrations of avoralstat in retinal tissue were measured at specific time points (1 month, 2 months, 3 months, and 6 months) after an intravitreal injection of an implant comprising of a PKal inhibitor and a polymer matrix. The polymer matrix was either 100% PEA III X50 or a mixture of PEA III X50 70% w/w and PLGA 752S 30% w/w. The composition of the implant was 60% w/w PEA III X50 and 40% w/w avoralstat or 42% w/w PEA III X50, 18% w/w PLGA 752S, and 40% w/w avoralstat. The results of the study are shown in FIG. 4. The tissue concentration of Avoralstat in the retina was above the IC₉₉ at all time points for both formulations. It was determined that avoralstat was released from the implant to the target tissue at an acceptable rate.

### Example 4

A polymer matrix comprising 100% PEA III X50 is combined with solid avoralstat and powdered and blended on a hot plate at 180 °C. A roller is then used to make a film on the 180 °C hot plate. The resulting film is then laminated at 170 °C with a tooling sheet to form implants sized for administration using a pharmaceutical syringe needle. After cooling, the implant is picked and loaded into the needle and administered to a subject with DME by IVT injection. It is determined that the avoralstat is released from the implant to the target tissues at an acceptable rate.

### Example 5

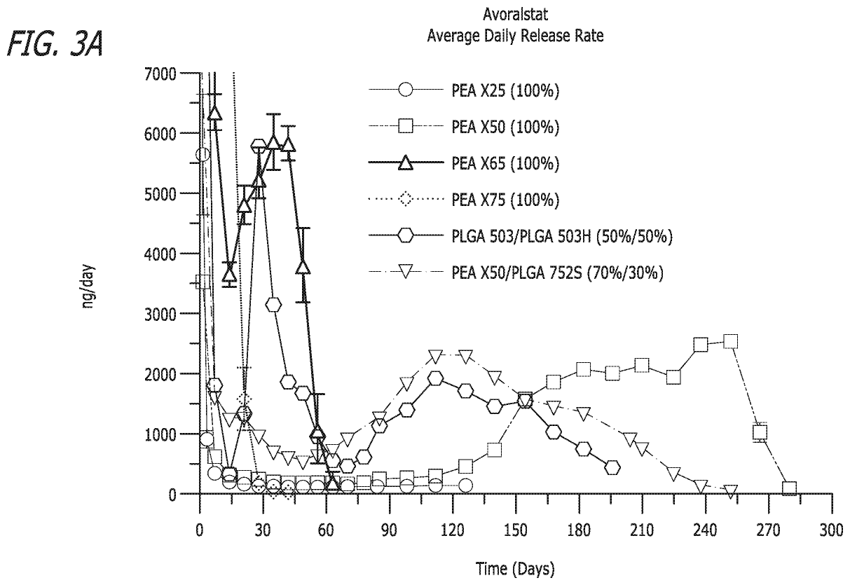

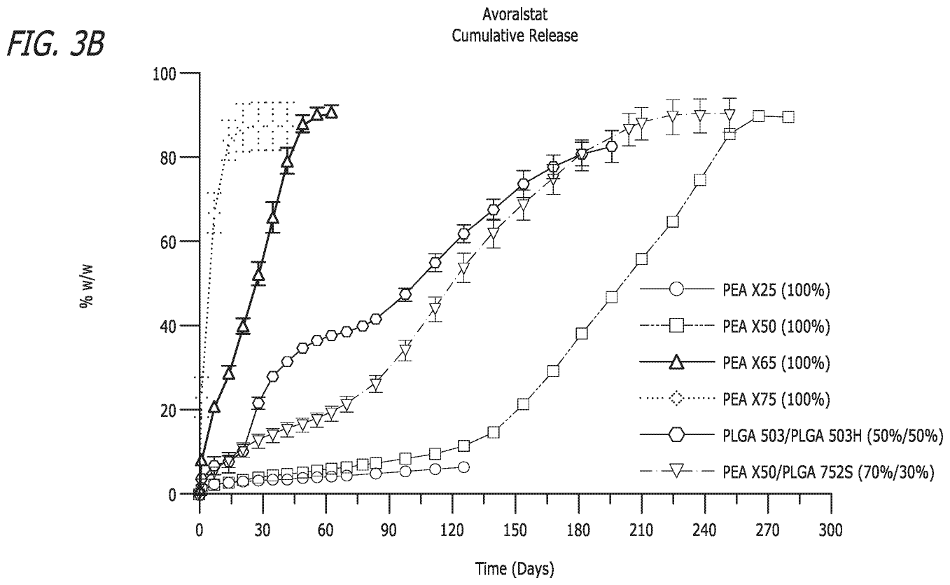

A polymer matrix comprising 100% PEA III X50 was combined with solid avoralstat and powdered and blended on a hot plate at 180 °C. A roller was then used to make a film on the 180 °C hot plate. The resulting film was then laminated at 170 °C with a tooling sheet to form implants sized for administration using a pharmaceutical syringe needle. The composition of the implant was 60% w/w PEA III X50 and 40% w/w avoralstat. After cooling, the implant was picked and evaluated in an in vitro dissolution kinetics study. For the in vitro dissolution study, each implant was placed into a closed vial with 3 mL of phosphate buffered saline (PBS) media. Closed vials were then placed in an incubator with controlled parameters (37 °C, 100 rpm) and drug release was monitored and analyzed by HPLC at specified intervals (every week for the first 10 weeks and every 2 weeks afterwards). Media in the vial was also replaced in parallel at each time point of analysis. Another implant, employing 100% PEA III X25 and solid avoralstat, was also evaluated in this study. The composition of the implant was 60% w/w PEA X25 and 40% w/w avoralstat. The results are shown in FIG. 3A and FIG. 3B. Overall, it was observed that, the 100% PEA III X50 implant, with higher % of acid functionality in the lysine group, showed faster drug release than the 100% PEA III X25 implant.

### Example 6

A polymer matrix comprising PEA III X50 and a first PLGA, optionally with a second PLGA different from the first, is combined with solid avoralstat and powdered and blended on a hot plate at 180 °C. A roller is then used to make a film on the 180 °C hot plate. The resulting film is then laminated at 170 °C with a tooling sheet to form implants sized for administration using a pharmaceutical syringe needle. After cooling, the implant is picked and loaded into the needle and administered to a subject with DME by IVT injection. It is determined that the avoralstat is released from the implant to the target tissues at an acceptable rate.

### Example 7

A polymer matrix comprising PEA III X50 and a PLGA 752S was combined with solid avoralstat and powdered and blended on a hot plate at 180 °C. A roller was then used to make a film on the 180 °C hot plate. The resulting film was then laminated at 170 °C with a tooling sheet to form implants sized for administration using a pharmaceutical syringe needle. The composition of the implant was 42% w/w PEA III X50, 18% PLGA 752S, and 40% w/w avoralstat. After cooling, the implant was picked and evaluated in an in vitro dissolution kinetics study. For the in vitro dissolution study, each implant was placed into a closed vial with 3 ml of appropriate media. Closed vials were then placed in an incubator with controlled parameters (37 °C, 100 rpm) and drug release was monitored and analyzed by HPLC at specified intervals (every week for the first 10 weeks and every 2 weeks afterwards). Media in the vial was also replaced in parallel at each time point of analysis. The results are shown in FIG. 3A and FIG. 3B. It was determined that the avoralstat was released from the implant polymer matrix comprising a PEA and PLGA mixture at an acceptable rate.

### Example 8

A polymer matrix comprising PEA III X25 and a first PLGA, optionally with a second PLGA different from the first, are combined with solid avoralstat and powdered and blended on a hot plate at 180 °C. A roller is then used to make a film on the 180 °C hot plate. The resulting film is then laminated at 170 °C with a tooling sheet to form implants sized for administration using a pharmaceutical syringe needle. After cooling, the implant is picked and loaded into the needle and administered to a subject with DME by IVT injection. It is determined that the avoralstat is released from the implant to the target tissues at an acceptable rate.

### Example 9

A polymer matrix comprising PEA III X25 and a PLGA, optionally with a PLA, are combined with solid avoralstat and powdered and blended on a hot plate at 180 °C. A roller is then used to make a film on the 180 °C hot plate. The resulting film is then laminated at 170 °C with a tooling sheet to form implants sized for administration using a pharmaceutical syringe needle. After cooling, the implant is picked and loaded into the needle and administered to a subject with DME by IVT injection. It is determined that the avoralstat is released from the implant to the target tissues at an acceptable rate.

### Example 10

A polymer matrix comprising PEA III X25 and a PLA, optionally with a PLGA, are combined with solid avoralstat and powdered and blended on a hot plate at 180 °C. A roller is then used to make a film on the 180 °C hot plate. The resulting film is then laminated at 170 °C with a tooling sheet to form implants sized for administration using a pharmaceutical syringe needle. After cooling, the implant is picked and loaded into the needle and administered to a subject with DME by IVT injection. It is determined that the avoralstat is released from the implant to the target tissues at an acceptable rate.

### Example 11

A polymer matrix comprising of 50% PLGA 503 and 50% PLGA 503H was combined with solid avoralstat and powdered and blended on a hot plate at 150 °C. A roller was then used to make a film on the 150 °C hot plate. The resulting film was then laminated at 140 °C with a tooling sheet to form implants sized for administration using a pharmaceutical syringe needle. The composition of the implant was 30% w/w PLGA 503, 30% PLGA 503H, and 40% w/w avoralstat. After cooling, the implant was picked and loaded into the needle and evaluated in an in vitro dissolution kinetics study. For the in vitro dissolution study, each implant was placed into a closed vial with 3 mL of PBS media. Closed vials were then placed in an incubator with controlled parameters (37 °C, 100 rpm) and drug release was monitored and analyzed by HPLC at specified intervals (every week for the first 10 weeks and every 2 weeks afterwards). Media in the vial was also replaced in parallel at each time point of analysis. The results are shown in FIG. 3A and FIG. 3B. It was determined that the avoralstat was released from the implant at an acceptable rate.

### Example 12

A polymer matrix comprising of a first PLGA, optionally with a second PLGA is combined with solid avoralstat and powdered and blended on a hot plate at 150 °C. A roller is then used to make a film on the 150 °C hot plate. The resulting film is then laminated at 140 °C with a tooling sheet to form implants sized for administration using a pharmaceutical syringe needle. After cooling, the implant is picked and loaded into the needle and administered to a subject with DME by IVT injection. It is determined that the avoralstat is released from the implant to the target tissues at an acceptable rate.

### Example 13

A polymer matrix comprising of a first PLGA, optionally with a second PLA is combined with solid avoralstat and powdered and blended on a hot plate at 150 °C. A roller is then used to make a film on the 150 °C hot plate. The resulting film is then laminated at 140 °C with a tooling sheet to form implants sized for administration using a pharmaceutical syringe needle. After cooling, the implant is picked and loaded into the needle and administered to a subject with DME by IVT injection. It is determined that the avoralstat is released from the implant to the target tissues at an acceptable rate.

### Example 14

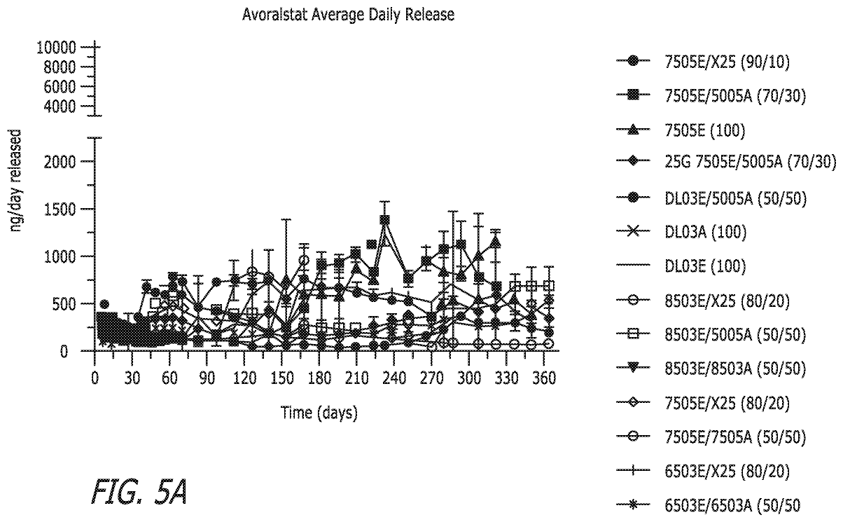

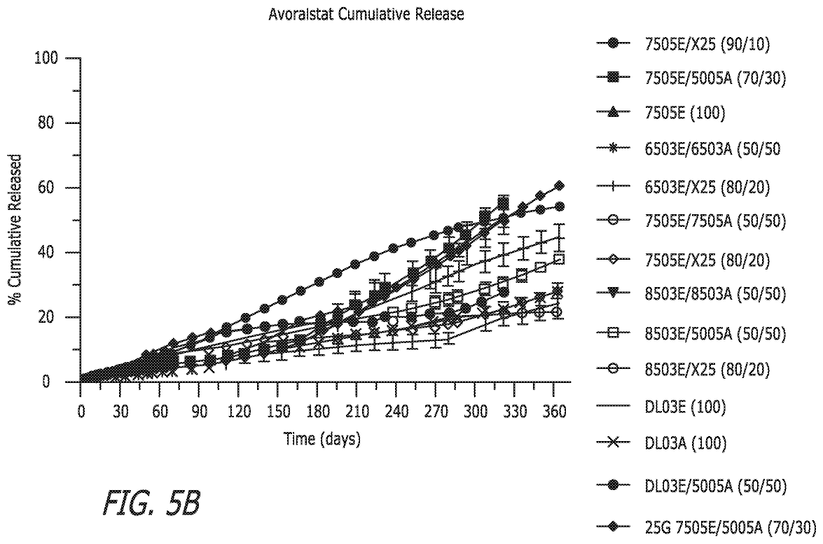

A polymer matrix comprising solid avoralstat and a composition of polymer(s) in Table 4 is powdered and blended on a hot plate at 150 °C. In these experiments, avoralstat is 40% by weight and the polymer(s) are 60% by weight. A roller is then used to make a film on the 150 °C hot plate. The resulting film is then laminated at 140 °C with a tooling sheet to form implants sized for administration using a pharmaceutical syringe needle. After cooling, the implant is picked and loaded into the needle and evaluated in an in vitro dissolution kinetics study. For the in vitro dissolution study, each implant was placed into a closed vial with 3 mL of PBS media. Closed vials were then placed in an incubator with controlled parameters (37 °C, 100 rpm) and drug release was monitored and analyzed by HPLC at specified intervals (every week for the first 10 weeks and every 2 weeks afterwards). Media in the vial was also replaced in parallel at each time point of analysis. The results are shown in FIG. 5A and FIG. 5B.

### Example 15

Implants comprising solid avoralstat and the composition of certain polymer(s) in Table 4 (as described in Example 14), along with other test articles, were studied in a rabbit DL-AAA model in vivo.

The following methods describe the induction, dosing, and evaluation of a rabbit DL-AAA ocular disease model for the assessment of therapeutic agents, including sustained release intraocular implants and injected test articles. The DL-AAA model provides a reproducible in vivo platform for quantifying retinal vascular leakage, neovascular pathology, and tissue drug distribution. Prior to study inclusion all animals were examined to confirm the absence of pre-existing ocular abnormalities. Rabbits were uniquely identified and stratified into experimental groups following baseline fluorescein angiography to ensure balanced distribution of induction phenotypes. Clinical observations and morbidity assessments were conducted throughout the study period.

For DL AAA induction, animals were pretreated with topical mydriatic agents and sedated using a combination of systemic sedative and inhaled anesthetic. Analgesia was administered to the ocular surface which was then aseptically prepared. A pars plana intraocular injection was performed using a fine 30 gauge needle positioned approximately 2-3 mm posterior to the limbus. Following injection, topical antimicrobial and lubricating agents were applied.

Test articles, including injectable formulations and/or implantable devices, were administered using a similar intraocular delivery procedure. For implant delivery, a syringe based delivery system was employed wherein the implant is expelled upon full depression of the plunger. Implants were administered without additional preparation. A pars plana intraocular injection of the implant and/or solution was performed using a fine 23, 25, or 30 gauge needle positioned approximately 2-3 mm posterior to the limbus. Following injection, topical antimicrobial and lubricating agents were applied.

Retinal vascular pathology was assessed longitudinally using fluorescein angiography. Animals were sedated and imaged using a confocal scanning laser ophthalmoscopy system using standardized orientation and intensity settings. Imaging was initiated concurrent with systemic fluorescein administration. Retinal vascular leakage was visualized and recorded at defined post injection intervals.

Quantitative retinal vascular leakage was assessed using digital image analysis. Standardized regions of interest were placed over secondary and tertiary retinal vasculature, with adjacent regions used to measure background signal. Thresholding algorithms were applied to isolate hyperfluorescent regions corresponding to vascular leakage. Corrected Total Regional Fluorescence (CTRF) was calculated by subtracting background adjusted signal from the threshold measurement region. Data were aggregated per rabbit and expressed as summary statistics. These methods provide a robust and quantifiable DL AAA model suitable for evaluating therapeutic efficacy, ocular pharmacokinetics, and disease modifying activity.

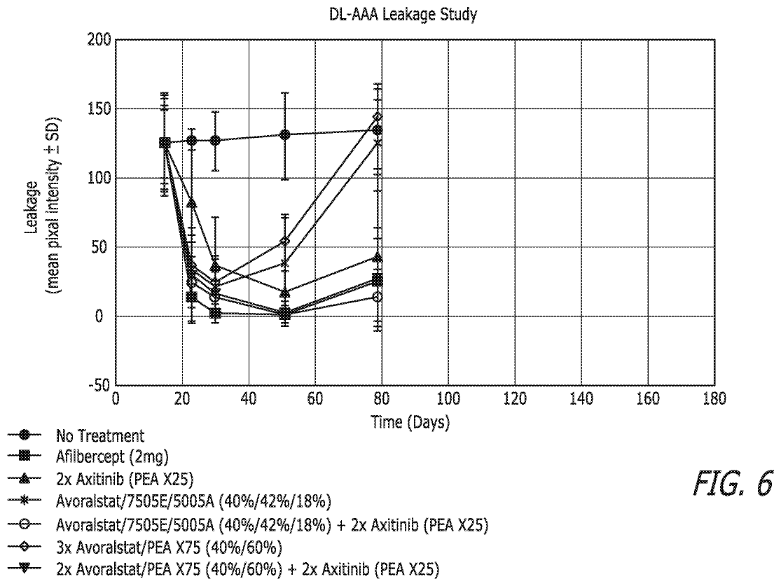

Certain compositions comprising avoralstat, with and without axitinib, are useful for reducing leakage for at least 90 days in the rabbit DL-AAA model. The compositions comprise 1) 60% w/w PLGA 7505E and 40% w/w avoralstat; 2) 42% w/w PLGA 7505E, 18% w/w PLGA 5005A, and 40% w/w avoralstat; 3) 54% w/w PLGA 7505E, 6% w/w PEA III X25, and 40% w/w avoralstat; and 4) 60% w/w PEA III X75 and 40% w/w avoralstat. Each of these implants comprising avoralstat may be administered concurrently with implant(s) comprising axitinib and PEA III X25. Results of these experiments are shown in FIG. 6

### Example 16

A study to determine target ocular tissue pharmacokinetic (PK) profiles of avoralstat implants was conducted in dutch belted rabbits. Concentrations of avoralstat in ocular tissue (iris ciliary body (ICB), retina, retinal pigment epithelium (RPE)/choroid, optic nerve head (ONH), and vitreous humor) were measured at specific time points (1 month, 2 months, 3 months, 4.5 and 6 months) after an intravitreal injection of an implant comprising avoralstat and a polymer matrix. Samples were processed and analyzed for avoralstat acid using a qualified LC-MS/MS analytical method. The implant compositions used in this study comprise three groups: 1A) 60% w/w PLGA 7505E and 40% w/w avoralstat; 2A) 42% w/w PLGA 7505E, 18% w/w PLGA 5005A, and 40% w/w avoralstat; 3A) 54% w/w PLGA 7505E, 6% w/w PEA III X25, and 40% w/w avoralstat. The results of the study are shown in FIGS. 7-11: FIG. 7 shows the results for group 1A; FIG. 8 shows the results for group 2A; and FIG. 9 shows the results for group 3A. Each of the groups succeeded in delivering concentrations of avoralstat to each of the ocular tissues. It was determined that the implants of group 2A), comprising 42% w/w PLGA 7505E, 18% w/w PLGA 5005A, and 40% w/w avoralstat, exhibited a consistently higher release of avoralstat as compared to the other two groups. The retinal and RPE/choroid concentrations of avoralstat (see FIG. 10 and FIG. 11, respectively) delivered from each of the implants was above the IC99 at all timepoints tested. Each group delivered avoralstat to the target tissues at an acceptable rate.

1. An intravitreal implant, comprising a bioerodible polymeric matrix and a plasma kallikrein (PKal) inhibitor selected from avoralstat or a pharmaceutically acceptable salt thereof, wherein the PKal inhibitor is dispersed within the bioerodible polymeric matrix.

2. The intravitreal implant of claim 1, wherein the PKal inhibitor is avoralstat.

3. The intravitreal implant of claim 1, wherein the PKal inhibitor is a pharmaceutically acceptable salt of avoralstat.

4. The intravitreal implant of claim 1, wherein the PKal inhibitor is a solid form of avoralstat or a pharmaceutically acceptable salt thereof.

5. The intravitreal implant of claim 1, comprising:
A) 60% w/w PEA III X50 and 40% w/w PKal inhibitor;
B) 42% w/w PEA III X50, 18% w/w PLGA 752S, and 40% w/w PKal inhibitor;
C) 30% w/w PLGA 503, 30% PLGA 503H, and 40% w/w PKal inhibitor;
D) 60% w/w PEA III X25 and 40% w/w PKal inhibitor;
E) 42% w/w PEA III X25, 18% w/w PLGA 752S, and 40% w/w PKal inhibitor;
F) 60% w/w PEA III X75 and 40% w/w PKal inhibitor;
G) 60% w/w PLGA 7505E and 40% w/w PKal inhibitor;
H) 42% w/w PLGA 7505E, 18% w/w PLGA 5005A, and 40% w/w PKal; or
I) 54% w/w PLGA 7505E, 6% w/w PEA III X25, and 40% w/w PKal inhibitor.

6. The intravitreal implant of claim 1, further comprising at least one compound selected, independently, from a vascular endothelial growth factor (VEGF) inhibitor or a vascular endothelial growth factor receptor (VEGFR) inhibitor.

7. The intravitreal implant of claim 1, further comprising one or more of axitinib, cabozantinib, fruquintinib, lenvatinib, pazopanib, ponatinib, regorafenib, sorafenib, sunitinib, or tivozanib, or a pharmaceutically acceptable salt thereof.

8. The intravitreal implant of claim 1, further comprising axitinib or a pharmaceutically acceptable salt thereof.

9. The intravitreal implant of claim 7, wherein the axitinib, cabozantinib, fruquintinib, lenvatinib, pazopanib, ponatinib, regorafenib, sorafenib, sunitinib, or tivozanib, or a pharmaceutically acceptable salt thereof, is dispersed within the bioerodible polymeric matrix.

10. The intravitreal implant of claim 1, further comprising one or more of aflibercept, bevacizumab, brolucizumab, faricimab, or ranibizumab, or a biosimilar thereof.

11. The intravitreal implant of claim 1, wherein the bioerodible polymeric matrix comprises a polyesteramide polymer, a poly(lactic-co-glycolic) acid polymer, or a polylactic acid polymer.

12. The intravitreal implant of claim 1, wherein the bioerodible polymeric matrix comprises a polyesteramide polymer and a poly(lactic-co-glycolic) acid polymer.

13. The intravitreal implant of claim 12, wherein the poly(lactic-co-glycolic) acid polymer comprises a mole fraction of lactic acid and a mole fraction of glycolic acid, wherein the mole fraction of lactic acid is not less than the mole fraction of glycolic acid.

14. The intravitreal implant of claim 11, wherein the polyesteramide polymer comprises an α-amino acid-diol-diester polyesteramide, wherein the α-amino acid-diol-diester polyesteramide comprises PEA III X25, PEA III X35, PEA III X50, PEA III X65, or PEA III X75, or a mixture thereof.

15. The intravitreal implant of claim 1, wherein the intravitreal implant is configured to release a therapeutically effective amount of the PKal inhibitor, wherein the therapeutically effective amount is, on a daily basis, an average of less than 0.6% of the total molar amount of the PKal inhibitor.

16. An intravitreal implant, which is a combination comprising an intravitreal implant comprising axitinib or a pharmaceutically acceptable salt thereof and an intravitreal implant comprising the intravitreal implant of claim 1.

17. A method of treating diabetic macular edema in a subject in need thereof, comprising intravitreal administration of the intravitreal implant of claim 1 to the subject.

18. A method of treating diabetic retinopathy, proliferative diabetic retinopathy, macular edema, age-related macular degeneration, or retinal vein occlusion in a subject in need thereof, comprising intravitreal administration of avoralstat or a pharmaceutically acceptable salt thereof and intravitreal administration of axitinib or a pharmaceutically acceptable salt thereof to the subject.

19. A method of treating diabetic retinopathy, proliferative diabetic retinopathy, macular edema, age-related macular degeneration, or retinal vein occlusion in a subject in need thereof, comprising intravitreal administration of the intravitreal implant of claim 16 to the subject.

20. The method of claim 17, wherein the subject experiences an at least 10% improvement in measurement of a sign or symptom of diabetic macular edema as compared to administration of dexamethasone or fluocinolone acetonide by intravitreal implant to the subject.

21. The method of claim 17, wherein the intravitreal implant releases a therapeutically effective amount of the PKal inhibitor for at least six months, wherein the therapeutically effective amount is, on a daily basis, an average of less than 0.6% of the total molar amount of the PKal inhibitor.

22. The method of claim 17, wherein the intravitreal implant releases a therapeutically effective amount of the vascular endothelial growth factor (VEGF) inhibitor or the vascular endothelial growth factor receptor (VEGFR) inhibitor for at least three months, wherein the therapeutically effective amount is, on a daily basis, an average of less than 1.2% of the total molar amount of the VEGF inhibitor or the VEGFR inhibitor.

23. The method of claim 17, wherein the intravitreal implant is prepared by a process comprising a thermal melt step.
